SEQUENCE LISTING (40 sequences)

SEQ ID NO: 1 - DNA, 100 bp, Artificial Sequence
  source: /note=“Description of Artificial Sequence Synthetic probe”
gcaacctgctccaaaagtgaacctcaaaaggacatcttcactggtgggccccacacaaagcttcttcatgagggaatcaaaggctttgggggctgtccaa

SEQ ID NO: 2 - DNA, 100 bp, Artificial Sequence
  source: /note=“Description of Artificial Sequence Synthetic probe”
acagaaaataggagcagagcagatcttgctctcccaaatccacaagaatcctccagtgcacctgacattgaactcttggaagcatctcctgccaaagcag

SEQ ID NO: 3 - DNA, 100 bp, Artificial Sequence
  source: /note=“Description of Artificial Sequence Synthetic probe”
ccaggctttcaagggttgccaatcttcaccgtcacctgatgtctgcatttccctgggttcctcatcagatggcctggtgtctttaatgctgattctcacc

SEQ ID NO: 4 - DNA, 100 bp, Artificial Sequence
  source: /note=“Description of Artificial Sequence Synthetic probe”
aacccaaaatccttgggattgtgcagattgtaatcgccatcatgaacctcagcataggaattatgatgataattgccactgtgtcgaccggtgaaatacc

SEQ ID NO: 5 - DNA, 100 bp, Artificial Sequence
  source: /note=“Description of Artificial Sequence Synthetic probe”
cctaggatattaacacttcattgcactggcttttgaggtgaatattagatttactgtaagtatgtaagtcaagcacttattaggtcaacaacacttcaac

SEQ ID NO: 6 - DNA, 100 bp, Artificial Sequence
  source: /note=“Description of Artificial Sequence Synthetic probe”
tggcaaatctatcttctgaaccactcatttctgtggtcttaatggctccaatttggggaccaataatgttcattgtctcaggatccctgtcaattgcagc

SEQ ID NO: 7 - DNA, 100 bp, Artificial Sequence
  source: /note=“Description of Artificial Sequence Synthetic probe”
acaactggcactaccatcgtggtgaaaacccagctcaagcatacccacaaatagagtcccacatcgaaactccaccacattactcaaggatactgtttct

SEQ ID NO: 8 - DNA, 100 bp, Artificial Sequence
  source: /note=“Description of Artificial Sequence Synthetic probe”
tgaatttacttagtgctctgggagcagcagctggaatcattctcctcatatttggcttccttctagatggggaattcatctgtggctattctccagatgg

SEQ ID NO: 9 - DNA, 100 bp, Artificial Sequence
  source: /note=“Description of Artificial Sequence Synthetic probe”
aaacaaaactaaataccacaaaaacaaatggaactataccgcagaagatatgtcttcatgataatgcagaaattccaaccatcacagggtagcaatgctt

SEQ ID NO: 10 - DNA, 100 bp, Artificial Sequence
  source: /note=“Description of Artificial Sequence Synthetic probe”
catgattccacaggtagtgaccaatgagaccatcacaacgatttcaccaaatggaatcaactttccccaaaaagacgagtcccagcctacccaacagagg

SEQ ID NO: 11 - DNA, 100 bp, Artificial Sequence
  source: /note=“Description of Artificial Sequence Synthetic probe”
agtttggctgctttagagcctgccttgcagcaatgtaagctggctttcacacaactagacacaacccaagatgcttatcatttctttagccctgagccat

SEQ ID NO: 12 - DNA, 100 bp, Artificial Sequence
  source: /note=“Description of Artificial Sequence Synthetic probe”
gcctccaatgtagcaagctctgttgttgccgtcattggcctcttcctcttcacctattgtctgatagccctggggagtgctttcccacactgtaactcag

SEQ ID NO: 13 - DNA, 100 bp, Artificial Sequence
  source: /note=“Description of Artificial Sequence Synthetic probe”
tgtcactacaacaatccaggtgtggtcattccaaatgtctatgcagcaaacccagtggtcatcccagaaccaccaaacccaataccaagttattccgaag

SEQ ID NO: 14 - DNA, 100 bp, Artificial Sequence
  source: /note=“Description of Artificial Sequence Synthetic probe”
cctaagacctctctgaaggttctctgtgtgatagccaacgttatcagcttgttctgcgcactggccggcttctttgtcattgccaaggacctcttcctgg

SEQ ID NO: 15 - DNA, 100 bp, Artificial Sequence
  source: /note=“Description of Artificial Sequence Synthetic probe”
tttcatggctgctaacacctgatgtaggtgcccatgagattcccatataacaaggcacacctcatgcattttgtgcaaaaggaaattcacaacaaggtga

SEQ ID NO: 16 - DNA, 100 bp, Artificial Sequence
  source: /note=“Description of Artificial Sequence Synthetic probe”
gtgggaaatcttggcttcgcagaggtttcggaggtttgtcttcaagatcattaagcacggagaactcagaatgttccagaatagactggcatttcagagg

SEQ ID NO: 17 - DNA, 100 bp, Artificial Sequence
  source: /note=“Description of Artificial Sequence Synthetic probe”
gaattcatcctcacctgcatagcctcacattttggatgccaggctgtctgctgcgcccattttcagaacatgacaatgttcccaaccatatttggtggca

SEQ ID NO: 18 - DNA, 100 bp, Artificial Sequence
  source: /note=“Description of Artificial Sequence Synthetic probe”
gctgtaatcgatgcattgaaggatgtggatacgtggtccttcgatgtcttttccctcaatgaggccagtggagatcatgcactgaagttcattttctatg

SEQ ID NO: 19 - DNA, 100 bp, Artificial Sequence
  source: /note=“Description of Artificial Sequence Synthetic probe”
caacaacggcggcatcgagtgtctacgcttcctcaatgagatcatctctgattttgactctctcctggacaatcccaaattccgggtcatcaccaagatc

SEQ ID NO: 20 - DNA, 100 bp, Artificial Sequence
  source: /note=“Description of Artificial Sequence Synthetic probe”
gcttgtggataatggagatcatgtgggttgaatttctaagagcgtgacctcctaagtctcacaaggaatcagagaatagctaaattgtccttcctgaggc

SEQ ID NO: 21 - DNA, 100 bp, Artificial Sequence
  source: /note=“Description of Artificial Sequence Synthetic probe”
caggtcatcactattggcaacgagcggttccgatgccctgaggctcttttccagccttccttcttgggtatggaatcctgtggcatccatgaaactacat

SEQ ID NO: 22 - DNA, 100 bp, Artificial Sequence
  source: /note=“Description of Artificial Sequence Synthetic probe”
aggttgtctcctgcgacttcaacagcaactcccactcttccaccttcgatgccggggctggcattgctctcaatgacaactttgtcaagctcatttcctg

SEQ ID NO: 23 - DNA, 100 bp, Artificial Sequence
  source: /note=“Description of Artificial Sequence Synthetic probe”
ccactagcttctcttctgttttgttccctatgtgtcgtgggtgggggagggggccacctgccttacctactctgagttgcctttagagagatgcattttt

SEQ ID NO: 24 - DNA, 100 bp, Artificial Sequence
  source: /note=“Description of Artificial Sequence Synthetic probe”
tgcccagcatgaccctgccctacagcctctgctcatatcttatgataaagctgcgtccaagagcattgtcaacaacggccactcctttaacgttgagttt

SEQ ID NO: 25 - DNA, 100 bp, Artificial Sequence
  source: /note=“Description of Artificial Sequence Synthetic probe”
atcgaagactatttcccggagtatgccaattatactgtccctgaagatgcaacaccagatgcgggagaagatcccaaagttacaagagcaaagttcttta

SEQ ID NO: 26 - DNA, 100 bp, Artificial Sequence
  source: /note=“Description of Artificial Sequence Synthetic probe”
caggcaagcgacgttccccaggacgggctgcttttgcacgggcccttcgcacgcaagcccaagcggattcgcacagccttctcgccctcgcagctgctgc

SEQ ID NO: 27 - protein, 247 aa, Mus musculus
MIPQVVTNETITTISPNGINFPQKDESQPTQQRQDSLKKHLKAEIKVIVAIQIMCAVTVLALGIILASVPPVPYFNSVFSVLLKSGYPFIGALFFIASGILSIITERKSTKPLVDASLTLNILSVSFAFVGIIIISVSLAGLHPASEQCKQSKELSLIEHDYYQPFYNSDRSECAVTKSILTGALSVMLIISVLELGLALLSAMLWLREGVLTSLRNRGSVKLNVSPGIVMNSLVFRMLQCSCLPSN

SEQ ID NO: 28 - protein, 244 aa, Mus musculus
MIPQVVTSETVAMISPNGMSLPQTDKPQPFHQWQDSLKKHLKAEIKVMAAIQIMCAVMVLSLGIILASVPSNLHFTSVFSVLLKSGYPFIGALFFIVSGILSIVTETKSTKILVDSSLTLNILSVSFAFMGIIIISVSLAGLHPASEQCLQSKELRPTEYHYYQFLDRNECFAAKSVLAGVFSLMLISTMLELGLAVLTAMLWWKQSHSNIPGNVMFLPHSSNNDSNMESKVLCNPSYEEQLVC

SEQ ID NO: 29 - protein, 247 aa, Mus musculus
MIPQVVTSETVTVISPNGISFPQTDKPQPSHQSQDSLKKHLKAEIKVMAAIQIMCAVMVLALGIILASVPSNLHFTSVFSILLESGYPFVGALFFAISGILSIVTEKKMTKPLVHSSLALSILSVLSALTGIAILSVSLAALEPALQQCKLAFTQLDTTQDAYHFFSPEPLNSCFVAKAALTGVFSLMLISSVLELGLAVLTATLWWKQSSSAFSGNVIFLSQNSKNKSSVSSESLCNPTYENILTS

SEQ ID NO: 30 - protein, 234 aa, Mus musculus
MRLQLGTKNIGWDCFPKDIIIHKREKTGHTYEKEDDLLIGVPSEATVLGTIQLLCALILASFGGILVSASYFNPEVSTTLISGYLFIGSLCFAIAGILSIISEKISTKPFALSSLASNVASSVVAVIGLFLFTYCLIALGSAFPHCNSEKKFLSLLSYLKSHHWKNEDKNCYLAYVGAMSALGMMLLFTVLEVFLAGYSSIFWWKQVYSNKPGGTFFLPQSQDHTQLVKSNLLQ

SEQ ID NO: 31 - protein, 236 aa, Mus musculus
MLVIQGTEQSALEAGYGAQQNGQPLYVNSHSWKRMTEKFLKGEPKILGIVQIVIAIMNLSIGIMMIIATVSTGEIPPSSVYIGYPIWGSLMFIISGSFSIVAGRRTTKGLVRSSLGLNITSSVFAFSGIVISSLSPGIYSFHVYYCTYRGSSEGCHMTLSILMGLDIVVVVLSVLEFCIGVSLSAFGCRVMCCNPGGVMIIMPSNPTKAETANPVTLQSGLMPPEHQERNVPENMH

SEQ ID NO: 32 - protein, 226 aa, Mus musculus
MQGQEQTTMAVVPGVAVPSKNSVMTSQMWNEKKEKFLKGEPKVLGVLQVMIAIINLSLGIIILTTLFSELPTSVMLMVPIWGSIMFIVSGSLSIAAGVTPTKCLIVASLTLNTITSVLAATASIMGVVSVAVGSQFPFRYNYTITKGLDVLMLIFNMLEFCLAVSVSAFGCEASCCNSREVLVVLPSNPVETVMAPPMTLQPLLPSEHQGTNVPGNVYKNHPGEIV

SEQ ID NO: 33 - protein, 226 aa, Mus musculus
MQGQEQTTMAVVPGGAPPSENSVMKSQMWNENKEKFLKGEPKVLGVVQVMIALINLSFGIIILANLSSEPLISVVLMAPIWGPIMFIVSGSLSIAAGVKPTKSLIISSLTLNTITSVLAATASIMGVVSVAVGSQFPFRYNYTITKGLDILMLILNMLEFCIAVSISAFGCKASCCNSSEVLVVLPSNPAVTVMAPPVTLQPLPPSEHQGKNVPENVYKNHSEEIV

SEQ ID NO: 34 - protein, 225 aa, Mus musculus
MQGLAQTTMAVVPGGAPPSENSVIKSQMWNKNKEKFLKGEPKVLGAIQVMIAFINFSLGIIIILNRVSERFMSVLLLAPFWGSIMFIFSGSLSIAAGVKPTKAMIISSLSVNTISSVLAVAASIIGVISVISGVFRQFRSQPAIASLDVLMTILNMLEFCIAVSVSAFGCKASCCNSSEVLVVLPSNSAVTVTAPPMILQPLPPSECQGKNVPENLYRNQPGEIV

SEQ ID NO: 35 - protein, 290 aa, Mus musculus
MNRPTAQGAVNLSGSKFSTAKSWEPEQERLTWQPGTVSMNTVTSPGPMANSVYVVAPPNSYPVVPGTVPQMPIYPSNQPQVHVISGHLPGLVPAMTEPPAQRVLKKGQVLGAIQILIGLVHIGLGSIMITNLFSHYTPVSLYGGFPFWGGIWFIISGSLSVAAETQPNSPCLLNGSVGLNIFSAICSAVGIMLFITDISISSGYIYPSYYPYQENLGVRTGVAISSVLLIFCLLELSIASVSSHFGCQVACCHYNNPGVVIPNVYAANPVVIPEPPNPIPSYSEVVQDSR

SEQ ID NO: 36 - protein, 271 aa, Mus musculus
MWERRGRGESAAGTAAVASRLRPPGSLNTMSAPPASNGVFVVIPPSNASGLRPPPAILPTSMCQPPGIMQFEESQLGAQAPRATQPPDLRPMETFLTGEPKALGTVQILIGLIHLGFGSVLLMVRRGHLGMLFIEGGVPFWGGACFIISGSLSVAAERNHTSCLLKSSLGTNILSAMAAFAGTAILLMDFGVTNWDVGRGYLAVLTIFTILEFFIAVIATHFGCQATRAQTNASVIFLPNAFGTDFNIPSPAVSPPPAYDNVAYMPKESSE

SEQ ID NO: 37 - protein, 263 aa, Mus musculus
MTSSQTTYPGTRGIPNPCPPTRSMAPHPPQPLNFLNVRNQVQTGQVSFITSPGIFPNTQLGRENVPTVNPALGAAISNVKDAAIALGGVQITIGLMHIGFGVVLGLLSTSYNMTWAFSSVAFIGGYPFWGGVFFIASGSLSISAFKEFSHCLMKSTLIINISSTIFAFVGVILFLCDLNINGYYYQDYWMVLSGRGIAGVLAIFSLLEFSIAGAMAYFAHQGILRCNRSVPVAPAVYVANPLMRESPSAPPIYDNIPDYATTQ

SEQ ID NO: 38 - protein, 265 aa, Mus musculus
MAGQAPTAVPGSVTGEVSRWQNLGPAQPAQKVAQPQNLVPDGHLEKALEGSDLLQKLGGFHIAIAFAHLAFGGYLISTVKNLHLVVLKCWYPLWGTGSFLVAGMAAMTTVTFPKTSLKVLCVIANVISLFCALAGFFVIAKDLFLEGPFPWPIWRPYPEPTTYIQRLELTLFCFTFLEIFLSGSTAITAYRMKRLQAEDKDDTPFVPDTPMELKGLSPGATTILIKMWPKDTPPLILAEPWPPALDCCWPLTPSIKLCFTRAQGP

SEQ ID NO: 39 - protein, 304 aa, Mus musculus
MHPEPAPPPSHSNPELPVSGGSSTSGSRRSRRRSGDGEPSGAPPLPPPPPAVSYPDWIGQSYSEVMSLNEHSMQALSWRKLYLSRAKLKASSRTSALLSGFAMVAMVEVQLDTDHDYPPGLLIVFSACTTVLVAVHLFALMISTCILPNIEAVSNVHNLNSVKESPHERMHRHIELAWAFSTVIGTLLFLAEVVLLCWVKFLPLKRQAGQPSPTKPPAESVIVANHSDSSGITPGEAAAIASTAIMVPCGLVFIVFAVHFYRSLVSHKTDRQFQELNELAEFARLQDQLDHRGDHSLTPGTHYA

SEQ ID NO: 40 - protein, 295 aa, Mus musculus
MEGHMLFFLLVVVVQFLTGVLANGLIVVVNAIDLIMWKKMAPLDLLLFCLATSRIILQLCILFAQLGLSCLVRHTLFADNVTFVYIINELSLWFATWLGVFYCAKIATIPHPLFLWLKMRISRLVPWLILASVVYVTVTTFIHSRETSELPKQIFISFSSKNTTRVRPAHALLSVFVFGLTLPFLIFTVAVLLLLSSLWNHSRQMRTMVGTREPSRHALVSAMLSILSFLILYLSHDMVAVLICTQGLHFGSRTFAFCLLVIGMYPSLHSIVLILGNPKLKRNAKMFIVHCKCCH

(19) United States
(12) Patent Application Publication
(10) Pub. No.: US 2019/0321443 A1
(43) Pub. Date: Oct. 24, 2019

(54) Modulators of MS4A activity

(71) Applicant: President and Fellows of Harvard College, Cambridge, MA (US)

(72) Inventors: Sandeep R. Datta, Newton, MA (US); Daniel M. Bear, Brookline, MA (US); Paul L. Greer, Brookline, MA (US)

(21) Appl. No.: 15/998,810

(22) PCT Filed: Feb. 16, 2017
(86) PCT No.: PCT/US2017/018132

§ 371 Date: Aug. 16, 2018

Related U.S. Application Data
(60) Provisional application No. 62/341,423, filed on May 25, 2016.
(60) Provisional application No. 62/295,717, filed on Feb. 16, 2016.

(51) Int. Cl. A61K 38/17; A61K 31/4965; C07K 16/28

(52) U.S. Cl. CPC A61K 38/1774; C07K 16/2887; A61K 31/4965

(57) ABSTRACT

Provided herein are methods of treating Alzheimer's disease, atopy, allergies, asthma, and a disease or disorder associated with neuroinflammation by modulating MS4A receptors. Also found herein are methods of modulating olfactory and gustatory properties of a substance by modulating MS4A receptors. Additionally, described herein are methods of identifying agents that are modulators of MS4A receptors.

### RELATED APPLICATIONS

This application claims the benefit of priority to U.S. Provisional Application No. 62/295,717, filed Feb. 16, 2016, and U.S. Provisional Application No. 62/341,423, filed May 25, 2016, each of which is hereby incorporated in its entirety.

### STATEMENT OF RIGHTS

This invention was made with government support under Grants 1DP2OD007109 and 1RO1DC011558 awarded by the National Institutes of Health. This invention was made with government support under Grant 2011122159 awarded by the National Science Foundation. The U.S. government has certain rights in the invention. This statement is included solely to comply with 37 C.F.R. § 401.14(a)(f)(4) and should not be taken as an assertion or admission that the application discloses and/or claims only one invention.

### BACKGROUND

Members of the MS4A (membrane-spanning 4-domain family, subfamily a) gene family are expressed on a wide variety of tissues, some of which have chemosensory function like the immune system or the gut. While certain members of the MS4A family have been implicated in a number of diseases, the function of the family is unknown and therefore it is unknown how this gene family contributes to physiology or disease. Accordingly, there is a need for methods and compositions for identifying agents that modulate the MS4A family, in order to target members of the MS4A family for the treatment of MS4A-associated diseases and disorders, and in order to influence the physiological function of MS4A family members.

### SUMMARY

In certain aspects, provided herein is a method of treating and/or preventing an MS4A-associated disease or disorder, such as Alzheimer's disease, atopy, a disease or disorder associated with neuroinflammation, allergies and/or asthma in a subject comprising administering to the subject an agent that modulates the activity of an MS4A receptor (e.g., an MS4A2 receptor, an MS4A4 receptor, an MS4A4A receptor, an MS4A4E receptor, an MS4A6 receptor, an MS4A6E receptor or an MS4A7 receptor). In certain aspects, provided herein is a method of modulating olfactory and gustatory properties of a substance (e.g., a cosmetic or food substance) comprising adding to the substance an agent that modulates the activity of an MS4A receptor (e.g., an MS4A4 receptor, an MS4A4A receptor, an MS4A4E receptor, an MS4A6 receptor, an MS4A6E receptor or an MS4A7 receptor). In some embodiments, the agent activates the MS4A receptor. In some embodiments, the agent inhibits activity of the MS4A receptor (e.g., an MS4A2 receptor, an MS4A4 receptor, an MS4A4A receptor, an MS4A4E receptor, an MS4A6 receptor, an MS4A6E receptor or an MS4A7 receptor). In some embodiments, the agent is a small molecule, a polypeptide (e.g., an MS4A protein or a fragment thereof, or an MS4A receptor ligand or fragment thereof), an antibody (e.g., an antibody specific for an MS4A receptor), an antibody-like molecule (e.g., an antibody-like molecule specific for an MS4A receptor), or a polynucleotide (e.g., encoding an MS4A protein or an inhibitory nucleic acid). In some embodiments, the small molecule is 2,5-dimethylpyrazine. In some embodiments, the small molecule is 3-aminopyrazine (3-AP). In some embodiments, the small molecule is tetramethylpyrazine. In certain aspects, provided herein is a method of determining whether a test agent is a modulator of an MS4A receptor (e.g., to select the agent as a potential therapeutic agent for the treatment of an MS4A-associated disease or disorder, such as Alzheimer's disease, atopy, a disease or disorder associated with neuroinflammation, allergies and/or asthma, or to select an agent capable of modulating olfactory and gustatory sensation), first by forming a test mixture comprising a test agent (e.g., a polynucleotide, a small molecule, an antibody, an antibody-like molecule, or a peptide), incubating the test mixture with cells expressing MS4A receptors and determining the level of calcium influx into the cell. In some embodiments, the level of calcium influx may be determined, for example, by depletion or extracellular calcium and concentration of ligand-dependent calcium transients as compared to a control mixture lacking the test agent. In some embodiments, a test agent that decreases or increases extracellular calcium and/or ligand-dependent calcium transients compared to the level of extracellular calcium and/or ligand-dependent calcium transients in a control mixture is a modulator of MS4A receptor activity. In some embodiments, the test agent is an antibody, an antibody-like molecule, a peptide, a small molecule or a polynucleotide. In some embodiments, the test agent and/or the MS4A receptor is linked to a detectable moiety. In some embodiments, the MS4A receptor is ectopically expressed. In some embodiments, the control mixture is substantially identical to the test mixture except that the control mixture does not comprise a test agent. In some embodiments, the control mixture is substantially identical to the test mixture except that the control mixture comprises a placebo. In some embodiments, cells are expressing GCaMP6s. In some embodiments, the test mixture may comprise long chain fatty acids, steroids, heterocyclic compounds, and/or pheromones. In some embodiments, the test agent is a member of a library of test agents. In some embodiments, the MS4A receptor is an MS4A2 receptor, an MS4A4 receptor, an MS4A4A receptor, an MS4A4E receptor, an MS4A6 receptor, an MS4A6E receptor or an MS4A7 receptor.

In some aspects, provided herein are methods of modulating an MS4A receptor in a cell comprising contacting the cell with an agent identified according to methods provided herein. In some embodiments, the cell may be a neuron, glial cell, an immune cell, a mast cell, an epithelial cell or a cell present in the respiratory tract. In some embodiments, the test agent is a polynucleotide, a small molecule, an antibody, an antibody-like molecule, or a polypeptide and or a member of a library of test agents. In some embodiments, the small molecule is 2,5-dimethylpyrazine. In some embodiments, the small molecule is 3-aminopyrazine (3-AP). In some embodiments, the small molecule is tetramethylpyrazine. In some embodiments, the MS4A receptor is an MS4A2 receptor, an MS4A4 receptor, an MS4A4A receptor, an MS4A4E receptor, an MS4A6 receptor, an MS4A6E receptor or an MS4A7 receptor.

### BRIEF DESCRIPTION OF THE DRAWINGS

FIG. 1 includes three sections, A, B, and C. A and B show the detected MS4As in GC-D+ and OMP+ sensory neurons. Section C shows analysis of expression of 12 Ms4a family members.

FIG. 2 includes two sections, A and B. Section A shows scatter plots of olfactory epithelial cells between wildtype and mutant mice. Section B shows the correlation of gene expression between RNAseq experimental samples.

FIG. 3 includes two sections, A and B. Section A illustrates chromosome 19 of Mus musculus, showing the tandem clustering of the entire Ms4a gene family in a single chromosomal location. Section B shows amino acid residues revealing that residues under positive selection primarily localize to the extracellular loops of MS4A proteins and bitter taste receptors.

FIG. 4 includes three sections, A, B, and C. Section A shows diversity and amino acid conservation of extracellular domains within the MS4As subfamilies. Section B shows multiple sequence alignments of the mouse MS4A proteins expressed in GC-D cells. Section C shows a phylogenetic tree of the MS4A receptor family.

FIG. 5 includes three sections, A, B, and C. Sections A, B and C show heat maps of the percent of cells expressing MS4A receptors that responded to each chemical across three independent experiments.

FIG. 6 includes four sections, A, B, C, and D. A and B show representative confocal images of HEK293 cells transfected with plasmids encoding GCaMP6S and N-terminal mCherry-fusion proteins of the indicated MS4A protein. Section C shows deconvolution of selected odorant mixtures and identifies monomolecular compounds that specifically activate MS4A receptors. Section D shows GCaMP6s fluorescence versus time averaged across all cells.

FIG. 7 includes three sections, A, B, and C. Section A shows single molecule fluorescent in situ hybridization of dissociated olfactory epithelial cells. Section B shows Ms4a probes (other than negative controls Ms4a1, Ms4a2, Ms4a5) give a significantly higher proportion of positive cells than negative controls. Section C shows cells labeled with probes against the two indicated Ms4a family members.

FIG. 8 includes three sections, A. B and C. Section A shows RNAscope assays of dissociated olfactory epithelial cells. Section B shows a graphical representation of necklace OSNs with one or more fluorescent puncta for each Ms4a and Olfr probe. Section C shows images of Car2+ cells co-labeled with additional Ms4a probe pairs.

FIG. 9 has four sections, A, B, C, and D. Section A shows anti-MS4A4B antibody stains of anti-PDE2A+ and OMP-IRES-GFP+ cells in sections of the olfactory epithelium. Section B shows immunostaining with antibodies against five different MS4A family members. Section C shows anti-MS4A antibody labeling of dendritic knobs. Section D shows anti-MS4A4B and anti-MS4A7 antibody staining of necklace glomeruli.

FIG. 10 includes two sections, A and B. Section A shows HEK293T cells stained with the indicated anti-MS4A antibody. Section B shows the staining of MS4A proteins treated with antigenic peptide.

FIG. 11 includes two sections, A and B. Section A shows cul-de-sac regions of olfactory epithelia from Emx1-cre;GCaMP3 mice. Section B shows fluorescent traces extracted from a necklace cell in response to the indicated monomolecular odorant.

FIG. 12 includes two sections A and B. Section A shows the quantification of mRNA expression in GC-D cells relative to OMP cells using the single-molecule detection method Nanostring. Section B shows immunohistochemical analysis of sections prepared from the nasal epithelium of mice co-expressing an Emx1-cre allele and a Cre-dependent GCaMP3 reporter using antibodies against GCaMP and the necklace marker CAR2.

FIG. 13 includes three sections, A, B and C. Section A shows images of cul-de-sacs from mice exposed to the indicated odorant, immunostained for the necklace cell marker PDE2A and the neuronal activity marker phospho-S6 as well as quantification of the proportion of pS6+ necklace cells in mice exposed to each odorant. Section C shows representative images and quantification of phospho-S6 positive, virally infected OSNs exposed to the indicated odorant.

### DETAILED DESCRIPTION

In some aspects, provided herein are methods of preventing or treating an MS4A-associated disease or disorder, such as Alzheimer's disease, allergies, atopy, a disease or disorder associated with neuroinflammation or asthma in a subject comprising administering to the subject an agent that modulates MS4A receptors. In certain aspects, provided herein is a method of modulating olfactory or gustatory properties of a substance comprising adding to the substance an agent that modulates the activity of an MS4A receptor. In some aspects, described herein is a method of determining whether a test agent is a modulator of an MS4A receptor. In some embodiments, the test agent is a member of a library of test agents. In some embodiments, the MS4A receptor is an MS4A2 receptor, an MS4A4 receptor, an MS4A4A receptor, an MS4A4E receptor, an MS4A6 receptor, an MS4A6E receptor or an MS4A7 receptor.

### Definitions

For convenience, certain terms employed in the specification, examples, and appended claims are collected here.

As used herein, the term “administering” means providing a pharmaceutical agent or composition to a subject, and includes, but is not limited to, administering by a medical professional and self-administering. Such an agent can contain, for example, an MS4A modulator such as an antibody, antigen binding fragment thereof, an antibody-like molecule, or polypeptide described herein.

The term “agent” is used herein to denote a chemical compound, a small molecule, a mixture of chemical compounds and/or a biological macromolecule (such as a nucleic acid, an antibody, an antibody fragment, a protein or a peptide). Agents may be identified as having a particular activity by screening assays described herein below. The activity of such agents may render them suitable as a “therapeutic agent” which is a biologically, physiologically, or pharmacologically active substance (or substances) that acts locally or systemically in a subject.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains: and all stereoisomers of any of any of the foregoing.

As used herein, the term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable region (abbreviated herein as V_{H}) and a heavy chain constant region. Each light chain includes a light chain variable region (abbreviated herein as V_{L}) and a light chain constant region. The V_{H} and V_{L} regions can be further subdivided into regions of hypervariability, termed complementarity determining regions (CDR), interspersed with regions that are more conserved, termed framework regions (FR). The term “antibody” includes, for example, naturally occurring forms of antibodies, recombinant antibodies, single chain antibodies, monoclonal antibodies, polyclonal antibodies, chimeric antibodies, humanized antibodies, human antibodies, multispecific antibodies (e.g., bispecific antibodies), single-chain antibodies and antigen-binding antibody fragments. The term “antibody” also includes “antibody-like molecule”, such as fragments of the antibodies (e.g., antigen-binding fragments). The term “antibody” may also refer to an antibody mimetic. An antibody mimetic may refer to any compound that specifically binds to an antigen, and may be artificial peptides, proteins, nucleic acids, or small molecules.

The terms “antigen binding fragment” and “antigen-binding portion” of an antibody, as used herein, refers to one or more fragments of an antibody that retain the ability to bind to an antigen. Examples of binding fragments encompassed within the term “antigen-binding fragment” of an antibody include Fab, Fab′, F(ab′)₂, Fv, scFv, disulfide linked Fv, Fd, diabodies, single-chain antibodies, and other antibody fragments that retain at least a portion of the variable region of an intact antibody. These antibody fragments can be obtained using conventional recombinant and/or enzymatic techniques and can be screened for antigen binding in the same manner as intact antibodies.

As used herein, the term “monoclonal antibody” refers to an antibody obtained from a population of substantially homogeneous antibodies that specifically bind to the same epitope, i.e., the individual antibodies comprising the population are identical except for possible naturally occurring mutations that may be present in minor amounts. The modifier “monoclonal” indicates the character of the antibody as being obtained from a substantially homogeneous population of antibodies, and is not to be construed as requiring production of the antibody by any particular method.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The term “recombinant” polynucleotide means a polynucleotide of genomic, eDNA, semisynthetic, or synthetic origin which either does not occur in nature or is linked to another polynucleotide in a non-natural arrangement.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body.

As used herein, a therapeutic that “prevents” a disorder or condition refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. (1998) Science 282:63), and natural product extract libraries. In another embodiment, the compounds are small, organic non-peptidic compounds. In a further embodiment, a small molecule is not biosynthetic.

As used herein, the term “subject” means a human or non-human animal selected for treatment or therapy.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment. e.g., the administration of a drug, such that at least one symptom of the disease is decreased or prevented from worsening.

### MS4As

As described herein, MS4As are four pass membrane receptors that are localized in the plasma membrane and are responsible for sensing environmental cues. As used herein, the term “MS4A” or “MS4A receptor” refers to transmembrane proteins, e.g., eukarvotic proteins, e.g., mammalian proteins, that are known to be part of the MS4A protein family. In certain embodiments, the methods provided herein relate to agents that modulate the expression and/or activity of MS4A1. In humans, MS4A1 is encoded by the Ms4a1 gene. Exemplary human MS41 mRNA sequences are provided at NCBI accession numbers NG_023388.1, NM_021950.3, and NM_152866.2, which is hereby incorporated by reference. In certain embodiments, the methods provided herein relate to agents that modulate the expression and/or activity of MS4A2. In humans, MS4A2 is encoded by the Ms4a2 gene. Exemplary human MS42 mRNA sequence is provided at NCBI accession number KR712129.1, which is hereby incorporated by reference. In certain embodiments, the methods provided herein relate to agents that modulate the expression and/or activity of MS4A4. In humans, MS4A4 is encoded by the Ms4a4 gene. Exemplary human MS4A4 mRNA sequence is provided at NCBI accession number AB013102.1 which is hereby incorporated by reference. In certain embodiments, the methods provided herein relate to agents that modulate the expression and/or activity of MS4A6. In humans, MS4A6 is encoded by the Ms46 gene. Exemplary human MS4A6 mRNA sequence is provided at NCBI accession number AB013104.1, which is hereby incorporated by reference. In certain embodiments, the methods provided herein relate to agents that modulate the expression and/or activity of Ms4A7. In humans, MS4A7 is encoded by the Ms4a7 gene. Exemplary human MS4A7 mRNA sequence is provided at NCBI accession number AB026043.1, which is hereby incorporated by reference. In certain embodiments, the methods provided herein relate to agents that modulate the expression and/or activity of MS4A8. MS4A8 is encoded by the Ms4a8 gene. Exemplary MS4A8 mRNA sequence is provided at NCBI accession number AB026044.1, which is hereby incorporated by reference. In certain embodiments, the methods provided herein relate to agents that modulate the expression and/or activity of Ms4A10. In humans, MS4A10 is encoded by the Ms410 gene. Exemplary human MS4A10 mRNA sequence is provided at NCBI accession number AB026046.1, which is hereby incorporated by reference. In certain embodiments, the methods provided herein relate to agents that modulate the expression and/or activity of MS4A13. In humans, MS4A13 is encoded by the Ms4a13 gene. Exemplary humans, MS4A13 mRNA sequences are provided at NCBI accession numbers KJ900785.1 and HF583583.1 which is hereby incorporated by reference. In certain embodiments, the methods provided herein relate to agents that modulate the expression and/or activity of MS4A15. In humans, MS4A15 is encoded by the Ms415 gene. Exemplary human MS4A15 mRNA sequence is provided at NCBI accession number AB026046.1, which is hereby incorporated by reference. In certain embodiments, the methods provided herein relate to agents that modulate the expression and/or activity of MS4A15. In humans, MS4A15 is encoded by the Ms4a15 gene. Exemplary human MS4A15 mRNA sequences are provided at NCBI accession numbers AY584608.1 and AY584609.1, which is hereby incorporated by reference. Variants of MS4A proteins can be produced by standard means, including site-directed and random mutagenesis.

### Modulators of MS4A Activity

In certain embodiments, the methods relate to an isolated small molecule capable of modulating (e.g. activating or inhibiting) the MS4A receptor. The isolated small molecules may be known odorants (e.g. long chain fatty acids, steroids, pheromones, or heterocyclic compounds), or a small molecule from a library of test molecules. In certain embodiments the small molecule is not a long chain fatty acid, a steroid, a pheromone, or a heterocyclic compound. As used herein, a small molecule modulates an MS4A receptor and alters the level of calcium influx into the cell, wherein the level of calcium influx is determined by extracellular calcium and levels of ligand-dependent calcium transients. Exemplary examples of small molecule ligands include saturated fatty acids, unsaturated fatty acids (e.g., decanoic acid, docosanoic acid, dodecanoic acid, eicosanoic acid, hexanoic acid, myristic acid, octadecanoic acid, octanoic acid, palmitic acid), steroids (e.g., 4-Androsten-17alpha-ol-3-one sulphate, 5-Androsten-3Beta 17Beta-diol disulphate, 1,3,5(10)-Estratrien-3 17Beta-diol disulphate, 1,3,5(10)-Estratrien-3 17alpha-diol 3-sulphate, 5alpha-pregnen-3alpha-ol-20-one sulphate, 5beta-pregnen-3beta-ol-20-one sulphate, 4-pregnan-11beta 21-diol-3 20-dione 21-sulphate, 4-pregnen-21-ol-3 20-ione glucosiduronate, 1,3,5(10)-Estratrien-3 17Beta-diol 3-sulphate, 4-pregnen-11beta 17,21-triol3 20-dione 21-sulphate), and compounds with nitrogenous rings (e.g., 2,5-dimethylpyrazine, 2,6-dimethylpyrazine, 2,3-dimethylpyrazine, indole, nicotine, pyrrolidine, pyridine, quinolone). In some embodiments, the small molecule is 2.5-dimethylpyrazine. In some embodiments, the small molecule is 3-aminopyrazine (3-AP). In some embodiments, the small molecule is tetramethylpyrazine. See Table 3 in Exemplification for additional examples of potential small molecule ligands.

Certain embodiments of the present invention relate to methods of modulating MS4A receptor activity. These methods include administering an agent that decreases the activity and/or expression of MS4A, and/or prevents the binding of ligands to MS4A receptors. Agents which may be used to modulate the activity of MS4A include antibodies, antibody-like molecules, pheromones, proteins, peptides, small molecules and inhibitory RNA molecules, e.g., siRNA molecules, shRNA, ribozvmes, and antisense oligonucleotides specific for MS4A receptors.

In some embodiments, the agent is an antibody (e.g. antibody-like molecule, monoclonal antibodies, polyclonal antibodies, chimeric antibodies, humanized antibodies, human antibodies, multispecific antibodies, single-chain antibodies and antigen-binding antibody fragments).

In some embodiments, any agent that modulates MS4A receptors can be used to practice the methods provided herein. Such agents can be those described herein, those known in the art, or those identified through screening assays (e.g. the screening assays described herein).

### Methods of Identifying Modulators of MS4A Receptor Activity

In certain aspects, provided herein is a method of determining whether a test agent is a modulator of an MS4A receptor (e.g., to select the agent as a potential therapeutic agent for the treatment of an MS4A-associated disease or disorder, such as Alzheimer's disease, atopy, allergies and/or asthma, or to select an agent capable of modulating olfactory or gustatory properties of a substance), first by forming a test mixture comprising a test agent (e.g., a polynucleotide, a small molecule, an antibody, an antibody-like molecule, or a peptide), incubating the test mixture with cells expressing MS4A receptors and determining the level of calcium influx into the cell. In some embodiments, the level of calcium influx may be determined, for example, by depletion or extracellular calcium and concentration of ligand-dependent calcium transients as compared to a control mixture lacking the test agent. In some embodiments, a test agent that decreases or increases extracellular calcium and/or ligand-dependent calcium transients compared to the level of extracellular calcium and/or ligand-dependent calcium transients in a control mixture is a modulator of MS4A receptor activity. In some embodiments, the test agent is an antibody, an antibody-like molecule, a peptide, a small molecule or a polynucleotide. In some embodiments, the test agent and/or the MS4A receptor is linked to a detectable moiety. In some embodiments, the MS4A receptor is ectopically expressed. In some embodiments, the control mixture is substantially identical to the test mixture except that the control mixture does not comprise a test agent. In some embodiments, the control mixture is substantially identical to the test mixture except that the control mixture comprises a placebo. In some embodiments, cells are expressing GCaMP6s. The test mixture may comprise long chain fatty acids, steroids, heterocyclic compounds, and/or pheromones.

In some embodiments, the test agent is a member of a library of test agents. In some embodiments, assays used to identify agents useful in the methods include a reaction between MS4A receptors and one or more assay components. The other components may be either a test compound (e.g. the agent), or a combination of test compounds. Agents identified via such assays, may be useful, for example, for preventing or treating Alzheimer's disease, allergies, atopy, asthma, or MS4A associated diseases, or may be useful for modulating olfactory or gustatory sensation. In some aspects, provided herein are methods of treating or preventing Alzheimer's disease atopy, allergies, and/or asthma in a subject comprising administering to the subject the test agent identified using the methods of identifying modulators of MS4As. In some aspects, provided herein are methods of modulating olfactory or gustatory properties of a substance comprising adding to the substance the test agent identified in using the methods of identifying modulators of MS4As.

In some embodiments, the test agent (e.g. a polypeptide, a polynucleotide, a RNA molecule, or a small molecule) or MS4A receptor is linked to a detectable moiety. As used herein, a detectable moiety may comprise a test agent or MS4A receptor of the present invention linked to a distinct polypeptide or moiety to which it is not linked in nature. For example, the detectable moiety can be fused to the N-terminus or C-terminus of the test agent either directly, through a peptide bond, or indirectly through a chemical linker.

Agents useful in the methods of the present invention may be obtained from any available source, including systematic libraries of natural and/or synthetic compounds. Agents may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see. e.g., Zuckermann et al., 1994, J. Med. Chem. 37:2678-85); spatially addressable parallel solid phase or solution phase libraries; synthetic library methods requiring deconvolution: the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059: Carell et al. (1994) Angew. Chem. Int. Ed. Engl. 33:2061; and in Gallop et al. (1994) J. Med. Chem. 37:1233.

Libraries of agents may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390: Devlin, 1990, Science 249:404-406: Cwirla et al. 1990. Proc. Natl. Acad Sci. 87:6378-6382: Felici, 1991, J. Mol. Biol. 222:301-310; Ladner, supra.).

Agents useful in the methods of the present invention may be identified, for example, using assays for screening candidate or test compounds which modulate the activity of MS4A receptors. For example, candidate or test compounds can be screened for the ability to alter calcium influx in a population of cells expressing MS4As. In some embodiments, the MS4As are endogenously expressed. In some embodiments, the MS4As are ectopically expressed. As described herein, the test compound is in a test mixture.

The basic principle of the assay systems used to identify compounds that modulate the activity of MS4A receptors involves preparing a test mixture containing test agents under conditions and for a time sufficient to allow the test agents to modulate the MS4A receptor.

In order to test an agent for modulatory activity, the reaction mixture is prepared in the presence and absence of the test compound. The test compound can be initially included in the reaction mixture, or subsequently added at a later time. Control mixtures are incubated without the test compound or with a placebo. The calcium influx may then be tested. A change in calcium influx, as measured by extracellular calcium and calcium transients, in the test mixture, but less or no such change in the control mixture indicates the test compound is a modulator of an MS4A receptor. The assay for compounds that modulate MS4A activity may be conducted with isolated test agent or pooled test agents. Pooled test agents comprise a test mixture with one or more test agents. The order of addition of test agents may be varied. For example, cells may be co-transfected as described above with a plasmid encoding GCaMP6s and either a plasmid encoding one of the MS4A proteins. Mixtures of chemicals or agents are added at consistent or varied concentration. Cells are then analyzed for fluorescence corresponding to calcium influx.

### Polypeptides

In certain embodiments, provided herein is isolated polypeptides capable of modulating the activity of an MS4A receptor. The isolated polypeptides may be an MS4A receptor (e.g., a soluble MS4A receptor), an MS4A receptor ligand, or a fragment thereof. Such polypeptides can be useful, for example, for inhibiting or activating an MS4A receptor and for identifying and/or generating agents that specifically bind to an MS4A receptor. In some embodiments the polypeptide comprises no more than 100, 90, 80, 70, 60, 50, 40, 30, 25 or 20 consecutive amino acids of a known MS4A ligand.

In some embodiments, the polypeptide described herein is able to bind to an MS4A receptor. In some embodiments, the binding of the polypeptide to MS4A receptor alters calcium influx into the cell, therefore altering signaling pathways correlating with the pathogenesis of Alzheimer's disease, atopy, allergies, or asthma or correlated with olfactory or gustatory sensation. As used herein, a polypeptide binds to MS4A receptors and alter the level of calcium influx in a cell, wherein the level of calcium influx is determined by depletion extracellular calcium and decrease of ligand-dependent calcium transients. In some embodiments, the polypeptides can be isolated from cells or tissue sources by an appropriate purification scheme using standard protein purification techniques. In another embodiment, polypeptides are produced by recombinant DNA techniques. Alternatively, polypeptides can be chemically synthesized using standard peptide synthesis techniques.

In some embodiments, the MS4A receptors are ectopically expressed. In some embodiments, the test agent is a chimeric or fusion polypeptide. A fusion or chimeric polypeptide can be produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, Ausubel et al., eds., John Wiley & Sons: 1992). Moreover, many expression vectors are commercially available that already encode a fusion moiety.

The polypeptides described herein can be produced in prokaryotic or eukaryotic host cells by expression of polynucleotides encoding a polypeptide(s). Alternatively, such peptides can be synthesized by chemical methods. Methods for expression of heterologous polypeptides in recombinant hosts, chemical synthesis of polypeptides, and in vitro translation are well known in the art and are described further in Maniatis et al., Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11:255; Kaiser et al. (1989) Science 243:187: Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference.

### Inhibitory RNA Molecules

In some embodiments, provided herein are inhibitory RNA molecules for inhibiting MS4A receptor expression. In some embodiments, the inhibitory RNA molecules may be contacted with a cell or administered to an organism. Alternatively, constructs encoding these may be contacted with or introduced into a cell or organism. Antisense constructs, antisense oligonucleotides, RNA interference constructs or siRNA duplex RNA molecules can be used to interfere with activity of a receptor of interest, e.g., an MS4A receptor. Typically, at least 15, 17, 19, or 21 nucleotides of the complement of the MS4A mRNA sequence are sufficient for an antisense molecule. Typically, at least 19, 21, 22, or 23 nucleotides of a target sequence are sufficient for an RNA interference molecule. The RNA interference molecule may have a 2 nucleotide 3′ overhang. If the RNA interference molecule is expressed in a cell from a construct, for example from a hairpin molecule or from an inverted repeat of the desired Ms4A receptor sequence, then the endogenous cellular machinery will create the overhangs. Inhibitory RNA molecules can be prepared by chemical synthesis, in vitro transcription, or digestion of long dsRNA by Rnase III or Dicer. These can be introduced into cells by transfection, electroporation, or other methods known in the art. See Hannon, G J, 2002. RNA Interference, Nature 418: 244-251: Bemstein E et al., 2002, The rest is silence. RNA 7: 1509-1521; Hutvagner G et al., RNAi: Nature abhors a double-strand. Curr. Opin. Genetics & Development 12: 225-232; Brummelkamp, 2002, A system for stable expression of short interfering RNAs in mammalian cells. Science 296: 550-553; Lee N S, Dohjima T, Bauer G, Li H, Li M-J, Ehsani A. Salvaterra P, and Rossi J. (2002). Expression of small interfering RNAs targeted against HIV-1 rev transcripts in human cells. Nature Biotechnol. 20:500-505: Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A. Bemstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G. Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99(9):6047-6052.

Antisense or RNA interference molecules can be delivered in vitro to cells or in vivo, e.g., injected into tissues of a mammal. Typical delivery means known in the art can be used. For example, an interfering RNA can be delivered systemically using, for example, the methods and compositions described in PCT Application No: PCT/US09/036223, PCT/US09/061381 PCT/US09/063927, PCT/US09/063931 and PCT/US09/063933, each of which is hereby incorporated by reference in its entirety. In certain embodiments the siRNA is delivered locally. For example, when the siRNA described herein is used to treat asthma, delivery to the respiratory tract can be accomplished by inhalers. Alternatively, when the interfering RNA described herein is used to treat Alzheimer's disease, the interfering RNA can be delivered intravenously or parenterally.

### Polynucleotide/Nucleic Acid Molecules

Also provided herein are nucleic acid or polynucleotide molecules that encode the MS4A receptors, antibodies, antigen binding fragments thereof and/or polypeptides described herein. For example, the polynucleotide may encode an MS4A protein or fragment thereof, or the polynucleotide may be an inhibitory polynucleotide specific for an MS4A receptor. The nucleic acids may be present, for example, in whole cells, in a cell lysate, or in a partially purified or substantially pure form.

Nucleic acids described herein can be obtained using standard molecular biology techniques. For example, nucleic acid molecules described herein can be cloned using standard PCR techniques or chemically synthesized. For antibodies obtained from an immunoglobulin gene library (e.g., using phage or yeast display techniques), nucleic acid encoding the antibody can be recovered from the library.

In certain embodiments, provided herein are vectors that contain the isolated nucleic acid molecules described herein (e.g., an MS4A receptor). As used herein, the term “vector,” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby be replicated along with the host genome. Moreover, certain vectors are capable of directing the expression of genes. Such vectors are referred to herein as “recombinant expression vectors” (or simply, “expression vectors”).

In certain embodiments, provided herein are cells that contain a nucleic acid described herein (e.g., a nucleic acid encoding an antibody, antigen binding fragment thereof, antibody-like molecule, or polypeptide described herein). The cell can be, for example, prokaryotic, eukaryotic, mammalian, avian, murine and/or human. In certain embodiments the cell is a neuron. In certain embodiments the cell is a glial cell. In certain embodiments the cell is a GC-D cell. In certain embodiments the cell is an immune cell. In certain embodiments the cell is a mast cell. In certain embodiments the cell is a cell of the respiratory tract. In some embodiments, the cells express other GCaMPs. In certain embodiments, the nucleic acid is operably linked to a transcription control element such as a promoter. In some embodiments the cell transcribes the nucleic acid and thereby expresses an antibody, antigen binding fragment thereof, an antibody-like molecule, or polypeptide described herein. The nucleic acid molecule can be integrated into the genome of the cell or it can be extrachromosomal.

### Pharmaceutical Compositions

In certain embodiments provided herein is a composition, e.g., a pharmaceutical composition, containing at least one antibody, an antibody-like molecule, small molecule, polynucleotide or polypeptide capable of modulating an MS4A receptor described herein, formulated together with a pharmaceutically acceptable carrier. In some embodiments, the composition includes a combination of multiple (e.g., two or more) agents.

Pharmaceutical compositions can be administered in combination therapy, i.e., combined with other agents.

As described in detail below, the pharmaceutical compositions provided herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) inhalation, for example, through an inhaler; or (4) topical administration, for example, in the form of a cream of lotion.

Methods of preparing these formulations or compositions include the step of bringing into association an agent described herein with the carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions suitable for parenteral administration comprise one or more agents described herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Regardless of the route of administration selected, the agents, which may be used in a suitable hydrated form, and/or the pharmaceutical compositions, are formulated into pharmaceutically-acceptable dosage forms by conventional methods known to those of skill in the art.

Methods Disclosed herein are novel therapeutic methods of treatment or prevention of MS4A-associated diseases and/or disorders, including Alzheimer's disease, asthma, allergies or a disease or disorder associated with nueroinflammation. Additionally, provided herein are methods for modulating olfactory and gustatory sensation.

In some embodiments, provided herein are therapeutic methods of treating Alzheimer's disease or a disease or disorder associated with nueroinflammation, comprising administering to a subject, (e.g., a subject in need thereof), an effective amount of an agent that inhibits MS4A expression or activity or inhibits the binding of a ligand to an MS4A receptor.

The pharmaceutical compositions may be delivered by any suitable route of administration, including orally, nasally, as by, for example, a spray, rectally, intravaginal, parenterally, intracisternally and topically, as by powders, ointments or drops, including buccally and sublingually. In certain embodiments the pharmaceutical compositions are delivered generally (e.g., via oral or parenteral administration). In certain other embodiments the pharmaceutical compositions are delivered locally through injection.

The therapeutic described herein may be administered through conjunctive therapy. Conjunctive therapy includes sequential, simultaneous and separate, and/or co-administration of the active compounds in a such a way that the therapeutic effects of the first agent administered have not entirely disappeared when the subsequent agent is administered. In certain embodiments, the second agent may be co-formulated with the first agent or be formulated in a separate pharmaceutical composition.

In certain embodiments, provided herein are methods of modulating olfactory or gustatory sensation as well as therapeutic methods of treating Alzheimer's disease, atopy, allergies, asthma, or a disease or disorder associated with neuroinflammation that include administering to a subject (e.g., a subject in need thereof), an effective amount of an agent described herein. In certain embodiments, provided herein are therapeutic methods of treating atopy, allergies, or asthma that include administering to a subject (e.g., a subject in need thereof), an effective amount of an agent described herein. A subject in need thereof may include, for example, a subject who has been diagnosed with Alzheimer's disease, atopy, allergies, asthma, or a disease or disorder associated with neuroinflammation, a subject predisposed to Alzheimer's disease, atopy, allergies, asthma, or a disease or disorder associated with neuroinflammation, or a subject who has been treated for Alzheimer's disease, atopy, allergies, asthma, or a disease or disorder associated with neuroinflammation, including subjects that have been refractory to the previous treatment.

In certain embodiments, provided herein are therapeutic methods of treating Alzheimer's disease, allergies, asthma, atopy, or a disease or disorder associated with neuroinflammation, comprising administering to a subject, (e.g., a subject in need thereof), an effective amount of an agent described herein.

In certain embodiments, provided herein arc methods of modulating olfactory or gustatory properties of a substance that include adding to a substance (e.g. food or fragrance) an agent capable of modulating a MS4A receptor. In some embodiments, the substance is a food. In some embodiments, the substance is a cosmetic (e.g., perfume). In some embodiments, the substance is a beverage. In some embodiments, the subject is a personal hygiene product, (e.g., soap, toothpaste, shaving cream, aftershave, facial cleanser, shampoo, conditioner, tampons, menstrual pads, or deodorant). In some embodiments, the substance is a pharmaceutical composition or product. In certain embodiments, the pharmaceutical composition is formulated for topical delivery. In some embodiments, the substance or pharmaceutical composition is a cream or a lotion. It will be appreciated that the substances or pharmaceutical compositions described herein can be provided in any cosmetically and/or dermatologically suitable form, for example, an emulsion, a cream, a mousse, a gel, a foam, a lotion, a mask, an ointment, a pomade, a solution, a serum, a spray, a stick, a patch, or a towelette. For example, a substance for topical administration can be more or less fluid and have the appearance of a white or colored cream, of an ointment, of a milk, of a lotion, of a serum, of a paste, of a mousse or of a gel. It can, where appropriate, be applied to the skin in the form of an aerosol. It can also be present in solid form and, for example, be in the form of a stick. It can be used as a care product and/or as a skin makeup product. In some embodiments, the substance is a household cleaner (e.g., dish soap, laundry detergent, or dish washing detergent). In certain embodiments, the substances or pharmaceutical compositions described herein also contain other cosmetic and dermatological ingredients, such as hydrophilic or lipophilic gelatinizing agents, preservatives, antioxidants, solvents, surfactants, thickeners, perfumes, fillers, pigments, odor absorbers and coloring substances.

In certain embodiments, the substances or pharmaceutical compositions described herein also contain oils. Examples of oils that can be included in the substance or pharmaceutical composition described herein include without limitation: hydro carbonaceous oils of animal origin (e.g., perhydrosqualene), hydro carbonaceous oils of vegetable origin (e.g., liquid fatty acid triglycerides which comprise from 4 to 10 carbon atoms and the liquid fraction of karite butter), synthetic esters and ethers of fatty acids (e.g., the oils of the formulae R¹COOR² and R¹OR² in which R¹ represents the residue of a fatty acid comprising from 8 to 29 carbon atoms and R² represents a branched or unbranched hydrocarbon chain which contains from 3 to 30 carbon atoms, such as Purcellin's oil, isononyl isononanoate, isopropyl myristate, ethyl-2-hexyl palmitate, octyl-2-dodecyl stearate, octyl-2-dodecyl erucate, and isostearyl isostearate; hydroxylated esters such as isostearyl lactate, octylhydroxystearate, octyldodecyl hydroxystearate, diisostearylmalate, triisocetvl citrate, and heptanoates, octanoates and decanoates of fatty alcohols; polyol esters, such as propylene glycol dioctanoate, neopentylglycol diheptanoate and diethyleneglycol diisononanoate; and pentaerythritol esters, such as pentaerythrityl tetraisostearate), linear or branched hydrocarbons of mineral or synthetic origin (e.g., volatile or nonvolatile paraffin oils and their derivatives, petrolatum, polydecenes, and hydrogenated polyisobutene such as parleam oil), fatty alcohols having from 8 to 26 carbon atoms (e.g., cetyl alcohol and stearyl alcohol and their mixture octyldodecanol, 2-butyloctanol, 2-hexyldecanol, 2-undecylpentadecanol, oleic alcohol or linoleic alcohol), partially hydrocarbonaccous and/or siliconaceous fluorinated oils, silicone oils (e.g., volatile or nonvolatile polymethylsiloxanes (PDMS) which have a linear or cyclic siliconaceous chain and which are liquid or pasty at ambient temperature, in particular cyclopoly-dimethylsiloxanes (cyclomethicones) such as cyclohexasiloxane; polydimethylsiloxanes which comprise alkyl, alkoxy or phenyl groups which are pendent or at the end of the siliconaceous chain, with the groups having from 2 to 24 carbon atoms; phenylated silicones such as phenyltrimethicones, phenyldimethicones, phenyl-trimethylsiloxydiphenylsiloxanes, diphenyldimethicones, diphenylmethyldiphenyltrisiloxanes, 2-phenylethyltrimethylsiloxysilicates and polymethylphenylsiloxanes), and combinations thereof.

In some embodiments, emulsifiers and co-emulsifiers are included in the pharmaceutical compositions or substances. Examples of emulsifiers and co-emulsifiers described include, without limitation: OAV emulsifiers, such as esters of fatty acid and polycethylene glycol, in particular PEG-100 stearate, and esters of fatty acid and glycerol, such as glyceryl stearate, as well as W/O emulsifiers such as the oxyethylenated poly(methylcetyl)(dimethyl)-methylsiloxane or the mixture of ethylene glycol acetyl stearate and glyceryl tristearate.

Hydrophilic gelatinizing agents that can be included in the pharmaceutical compositions described herein include carboxyvinylic polymers (carbomer), acrylic polymers such as acrylate/alkyl acrylate copolymers, polyacrylamides, polysaccharides, natural gums and clays. Lipophilic gelatinizing agents may also be used such as modified clays (e.g., bentonites, metallic salts of fatty acids, hydrophobic silica and polyethylenes).

Examples of fillers that may be included in the pharmaceutical compositions described herein include, without limitation, pigments, silica powder, talc, starch which is crosslinked with octenylsuccinic anhydride, polyamide particles, polyethylene powders, microspheres based on acrylic copolymers, expanded powders such as hollow microspheres, silicone resin microbeads and combinations thereof.

Actual dosage levels of the active ingredients in the pharmaceutical compositions may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the compounds employed in the pharmaceutical composition at levels lower than that required in order to achieve the desired therapeutic effect and gradually increase the dosage until the desired effect is achieved.

### EXEMPLIFICATION

### Example 1: Transcriptional Profiling Reveals Expression of Ms4a Genes in Necklace Sensory Neurons

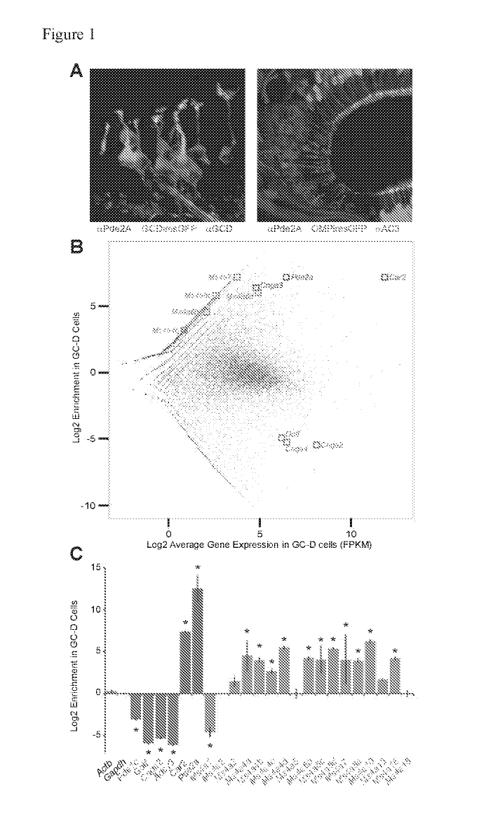

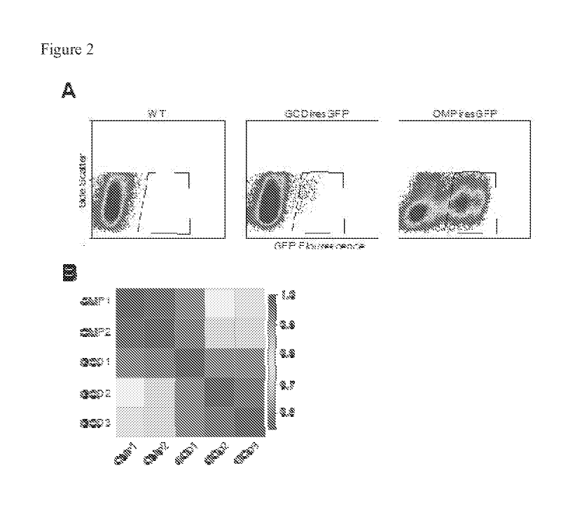

The Gucy2d-IRES-TauGFP allele marks necklace sensory neurons expressing PDE2A (blue) and GC-D (red) (FIG. 1, Section A). Pde2a+ necklace sensory neurons reside in caudal “cul-de-sac” regions of the main olfactory epithelium and do not express the Omp-IRES-GFP allele or the conventional OR signal transduction protein Adenyl Cyclase 3 (red). FIG. 1 plots the average enrichment versus expression for every sequenced mRNA transcript in GC-D+ and OMP+ sensory neurons. Each point on the graph is an mRNA with detectable RNAseq reads, with marker genes associated with GC-D cells (Car2, Pde2a, and Cnga3) and OMP cells (Go; Cnga2, and Cnga4) labeled in red and green, respectively; members of the Ms4a family that were reliably detected in these sequencing experiments are highlighted in blue (FIG. 1, Section B). Quantification of mRNA expression in GC-D cells relative to OMP cells using the single-molecule detection method Nanostring (FIG. 1, Section B). Marker genes for OMP cells (red bars) and GC-D cells (green bars) are enriched in the appropriate populations (FIG. 1, Section C). A comprehensive analysis of all annotated Ms4a genes indicates that 12 Ms4a family members are significantly enriched in GC-D cells relative to OMP OSNs (blue bars). A paired t-test revealed p<0.05. FIG. 2, Section A shows scatter plots of FAC sorted, dissociated olfactory epithelial cells from wild type mice, mice harboring the Gucy2d-IRES-TauGFP allele, or mice expressing the Omp-IRFS-GFP allele. The gate used to isolate ˜100% pure populations of fluorescent necklace OSNs or canonical OSNs is indicated. FIG. 2, Section B heat map of the correlation of gene expression between RNAseq samples, with warmer colors corresponding to more highly correlated gene expression.

### Example 2: Phylogenetic and Evolutionary Analysis of the Ms4a Gene Family Reveals Genomic Clustering and Positive Selection in Extracellular Domains

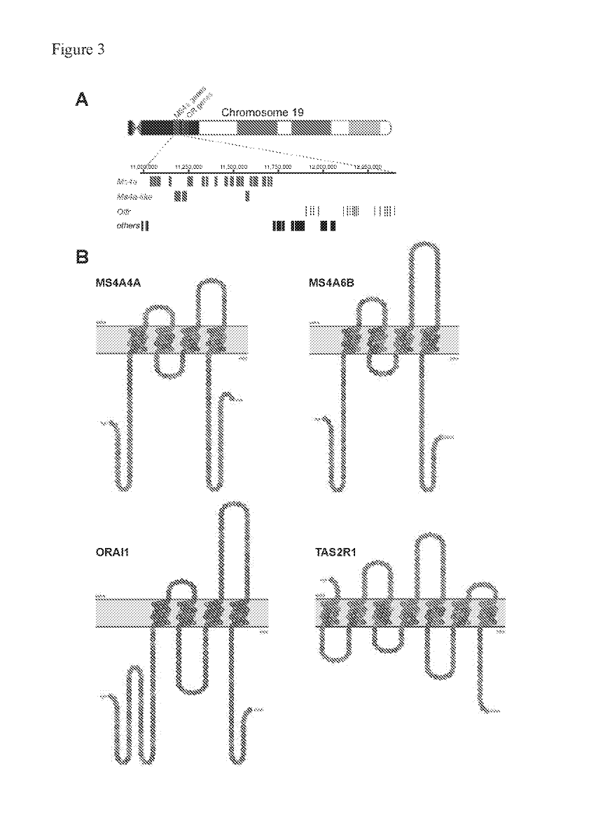

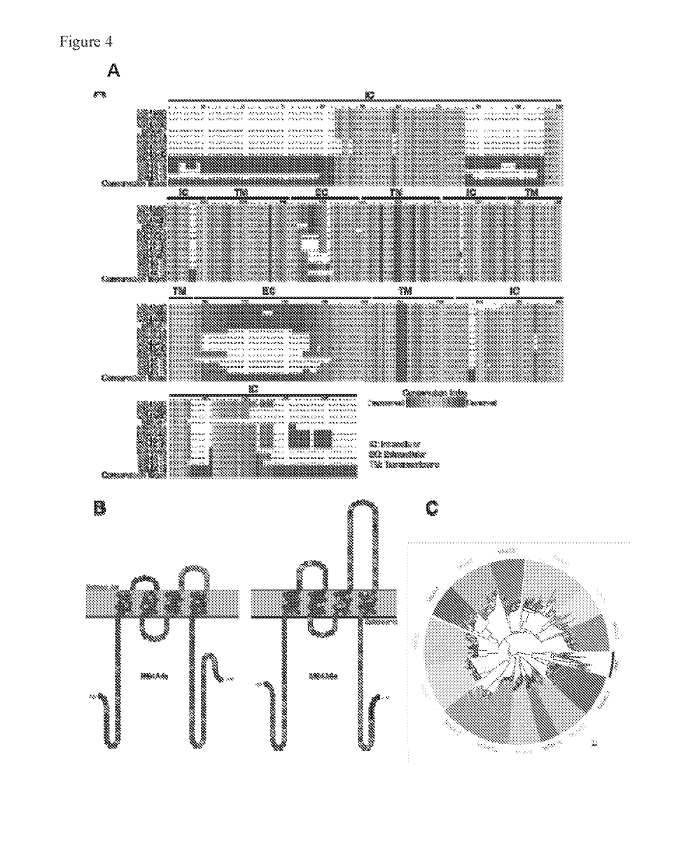

FIG. 3, Section A illustrates chromosome 19 of Mus musculus illustrating the tandem clustering of the entire Ms4a gene family (red) in a single chromosomal location. Immediately telomeric to the AM4a gene cluster resides a large group of conventional mammalian odorant receptor genes (blue). Primary sequences of Mus musculus MS4A4A, MS4A6C, ORAI1, and TAS2R1 arrayed along topographical representations of the proteins. The amino acid residues under strong purifying selection are shown in blue, whereas those under positive selection are shown in red (posterior probability>0.90), revealing that residues under positive selection primarily localize to the extracellular loops of MS4A proteins and bitter taste receptors (p=5.06×10⁻¹⁶ and p=6.76×10⁻⁷, respectively, hypergeometric test) (FIG. 3, Section B). FIG. 4 shows multiple sequence alignment of the mouse MS4A proteins expressed in GC-D cells. Residues that are more conserved are shown in warmer colors, whereas residues that are less conserved are depicted in colder colors. Conservation scores were determined using PRALINE. Firstly, FASTA format sequences of the indicated Mus musculus MS4A proteins were downloaded from the NCBI protein database and aligned using the PRALINE sequence alignment program on the Centre for Integrative Bioinformatics VU website using the default settings. Secondly, amino acid conservation across family members was scored using the PRALINE default settings where the least conserved amino acids were given a 0 score and the most conserved amino acids were assigned a 10 (Simossis and Heringa, 2005)). TOPCONS was used to determine the predicted topology of the MS4A family member that was used on the top line of the alignment. All topographical representations were generated using the Protter program and manually entering the topographical orientation of the MS4A protein as predicted by TOPCONS.

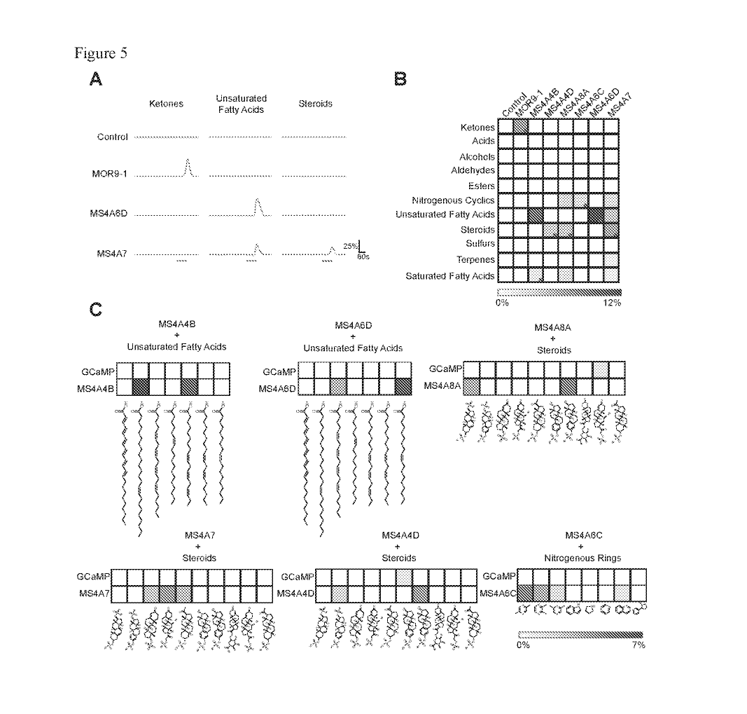

The intracellular (IC), extracellular (EC), and transmembrane (TM) regions of the proteins reveal the greatest sequence diversity in the extracellular domains, with additional diversity in the intracellular C-terminus (FIG. 4, Section A). Multispecies alignments of MS4A proteins from either the MS4A4 or MS4A6 subfamilies. Amino acid conservation was determined and heat-mapped as in FIG. 4. As with alignments of all GC-D-expressed MS4As, the extracellular domains within these subfamilies are more diverse than other regions of the MS4As (FIG. 5, Section B). A phylogenetic tree (FIG. 4, Section C) of the mammalian Ms4a gene family was generated using every Ms4a gene found in 37 representative taxa, which were selected to cover all major mammalian lineages (Table 1 below).

TABLE 1
List of taxa used for the phylogenetic reconstructions

| Group | Order | Family | Species | Common name |
|---|---|---|---|---|
| Monotremata | | Ornithorhynchidae | Ornithorhynchus anatinus | platypus |
| Marsupalia | | Dasyuridae | Sarcophilus harrisii | Tasmanian devil |
| Placentalia | Soricomorpha | Soricidae | Sorex araneus | common shrew |
| | Carnivora | Mustelidae | Mustela putorius furo | ferret |
| | | Ursidae | Ailuropoda melanoleuca | giant panda |
| | | Canidae | Canis lupus familiars | dog |
| | Chiroptera | Vespertilionidae | Myotis lucifugus | little brown bat |
| | Perissodactyla | Equidae | Equus ferus caballus | horse |
| | Artiodactyla | Suidae | Sus scrofa domesticus | pig |
| | | Bovidae | Bos taurus | cow |
| | | | Ovis aries | sheep |
| | Cetacea | Delphinidae | Tursiops truncatus | bottlenose dolphin |
| | | Lipotidae | Lipotes vexillifer | baiji |
| | Macroscelidea | Macroscelididae | Elephantulus edwardii | elephant shrew |
| | Afrosoricida | Tenrecidae | Echinops telfairi | lesser hedgehog tenrec |
| | Hyracoidea | Procaviidae | Procavia capensis | rock hyrax |
| | Cingulata | Dasypodidae | Dasypus novemcinctus | nine-banded armadillo |
| | Primates | Galagidae | Otolemur garnetti | northern greater galago |
| | | Callitrichidae | Callithrix jacchus | common marmoset |
| | | Hominidae | Gorilla gorilla | gorilla |
| | | | Pan troglodytes | chimpanzee |
| | | | Homo sapiens | human |
| | Scandentia | Tupaiidae | Tupaia chinensis | tree shrew |
| | Lagomorpha | Ochotonidae | Ochotona princeps | American pika |
| | | Leporidae | Oryctolagus cuniculus | rabbit |
| | Rodentia | Heteromyidae | Dipodomys ordii | kangaroo rat |
| | | Dipodidae | Jaculus jaculus | jerboa |
| | | Muridae | Mus musculus | mouse |
| | | | Rattus norvegicus | rat |
| | | Cricetidae | Cricetulus griseus | Chinese hamster |
| | | | Mesocricetus auratus | golden hamster |
| | | | Microtus ochrogaster | prairie vole |
| | | Sciuridae | Spermophilus | thirteen-lined ground |
| | | | tridecemlineatus | squirrel |
| | | Bathyergidae | Heterocephalus glaber | naked mole-rat |
| | | Chinchillidae | Chinchilla lanigera | chinchilla |
| | | Octodontidae | Octodon degus | degu |
| | | Caviidae | Cavia porcellus | guinea pig |

Every Ms4a gene was assigned to an MS4A subfamily and each subfamily is represented with a unique color to facilitate visualization within the circular phylogenetic tree. MS4A sequences from both Ensembl and NCBI databases were retrieved and imported them into Geneious v8 (Biomatters Ltd). 37 representative taxa from all the major mammalian lineages were chosen (Table 1). When a gene had more than one predicted isoform, the sequence that contained the longest open-reading frame was selected. Coding DNA sequences were translated, aligned with MAFFT v7.017 (Katoh and Standley, b) using the E-INS-i algorithm, the BLOSUM80 scoring matrix, and a gap-opening penalty of 1. For phylogenetic reconstruction of the multigene family tree, the OpenMPI version of MrBayes v3.2.1 (Ronquist et al., 2012) was used and the GTR+I+G model as determined by jModelTest 2.1.7 (Darriba et al., 2012). The final dataset consisted of 411 sequences and 447 characters corresponding to sites present in at least 75 percent of the aligned sequences. For individual gene tree reconstructions and evolutionary analyses, sequence subsets were extracted based on their group membership as predicted based on the multigene family tree. Sequences corresponding to each subset as above were aligned, and trimmed the resulting alignments to remove positions that contained gaps in the majority of sequences. The phylogenetic reconstruction was carried out using the OpenMPI version of RAxML v8 (Stamatakis, 2014).

To identify branches under episodic positive selection, the random-effects likelihood branch-site method (BS-REL) (Kosakovsky Pond et al., 2011) was used and implemented in the HyPhy package (Pond et al., 2005). The branch-site models allow the nonsynonvmous to synonymous substitution rate ratio ω (d/ds) to vary both among amino acid sites in the protein and across branches on the tree to detect positive selection affecting specific sites along particular lineages (Anisimova and Yang, 2007). Evidence for site-specific positive selection was identified in MS4A homologs using the codeml program in the PAML v4.8 software package (Anisimova and Yang, 2007). Different site models were compared, in which the evolutionary rate ω is allowed to vary among sites. Comparison of model pairs revealed M1a (neutral, codon values of ω fitted into two discrete site classes between 0 and 1) versus M2a (positive selection; similar to M1a but with one additional class allowing ω>1): M7 (neutral; values of ω following a beta distribution with ω=1 maximum) versus M8 (positive selection; similar to M7 but with one additional class allowing ω>1); and M8a (neutral; similar to M7 but with one fixed class with ω=1) versus M8. Multiple starting values of ω were chosen, and either the F3x4 or F61 model of codon frequencies. To evaluate whether the models allowing positive selection provided a significantly better fit to the data, likelihood ratio tests were used. Notably, the M1a-M2a comparison is more stringent and can lack power to detect signatures of diversifying selection compared to the M7-M8 models, which impose less constraints on the distribution of ω. Finally, the M8a vs. M8 comparison can be used to contrast the potential role of reduced purifying selection (or relaxation) versus positive selection. When the null model is rejected, the empirical Bayes procedure was used and implemented under model M8 to identify sites under positive selection (posterior probability ≥0.90). To identify sites that have experienced purifying selection (posterior probability ≥0.90), the Fast Unconstrained Bayesian AppRoximation (FUBAR) (Murrell et al., 2013) was used and as implemented in the HyPhy package. Consensus topology predictions were made using a standalone version of TOPCONS2.0 (Tsirigos et al., 2015). All computational analyses were run on the Odyssey cluster supported by the FAS Division of Science, Research Computing Group at Harvard University. Each Ms4a gene is represented as a line within this plot where the length of the line corresponds to the degree of evolutionary change within a lineage over time (the scale bar represents the number of substitutions per site). The Ms4a gene family cluster diversified through tandem duplication early in the evolution of mammals as illustrated by the presence of 10 homologs in the monotreme (platypus, light blue lines) and marsupial (Tasmanian devil, red lines) representatives, which contrasts the single copy of MS4A15 found in bird genomes (Zuccolo et al., 2010). Further extension of the family occurred during the evolution of placental mammals, with human and mouse genomes harboring 19 and 17 copies, respectively. The majority of MS4A subfamilies exhibit one-to-one orthologous pairs across species. By contrast, the MS4A4 and MS4A6 subfamilies, which are highly enriched in GCD neurons, demonstrate complex one-to-many and many-to-many paralogous relationships between species. It is noteworthy that 50% of the genes present in the bovid representatives are either lost or pseudogenized in cetacean lineages suggesting rapid gene turnover throughout evolution.

### Example 3: MS4A Proteins are Sufficient to Confer Responses to Small Molecule Odorants

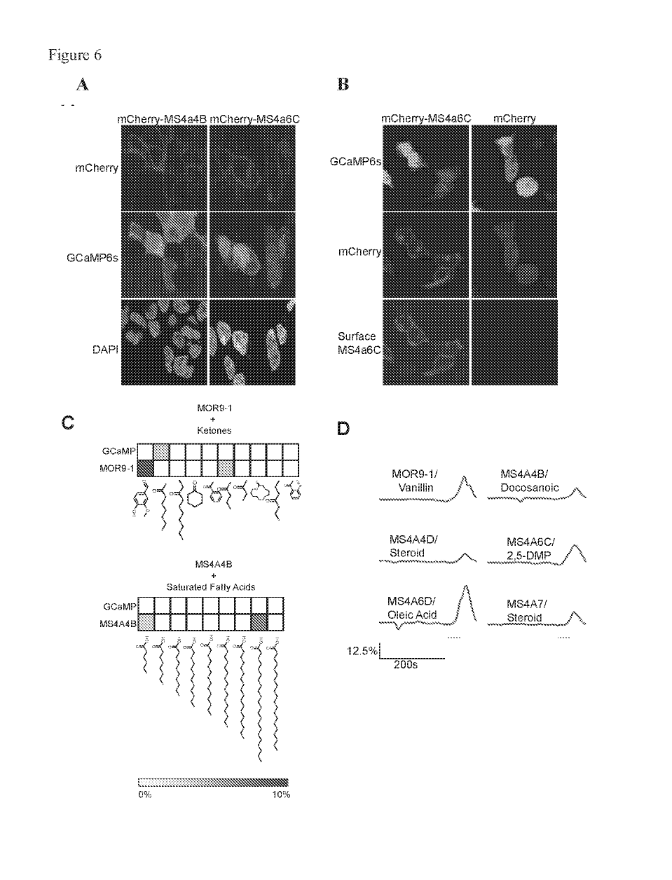

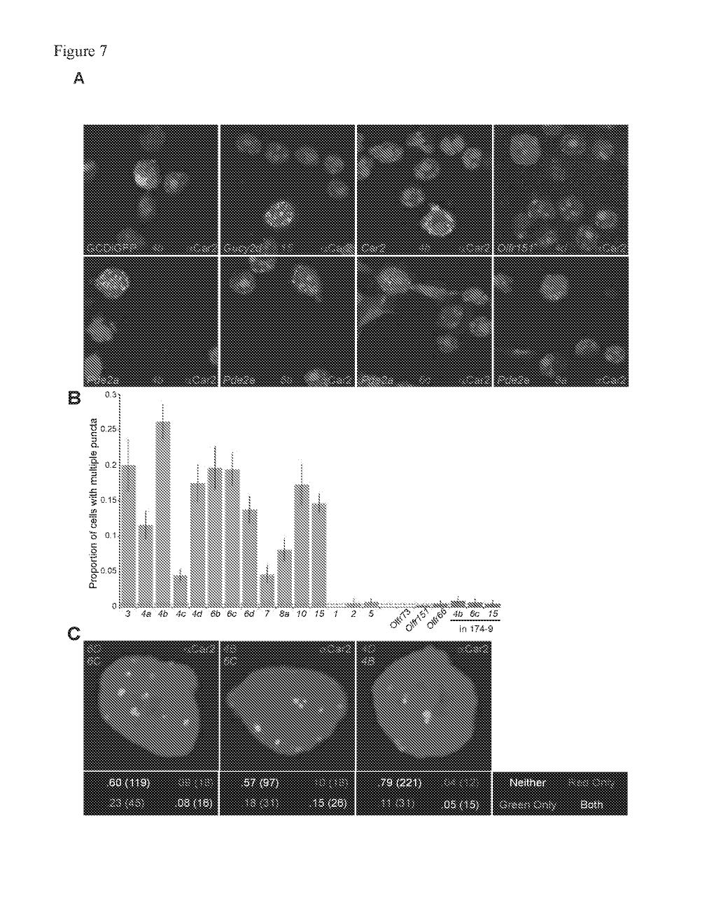

HEK293 cells were transfected with plasmids encoding the genetically encoded calcium reporter GCaMP6s and the indicated MS4A protein or GPCR mOR+G-protein; GCaMP6s fluorescence was measured as the indicated chemical mixtures were delivered in liquid phase (grey bars). Example traces of fluorescence intensity versus time derived from representative cells are shown. Control cells were transfected with GCaMP6s alone (FIG. 7, Section A). FIG. 7, Section B shows the responses of expressed MS4A protein/odor mixture pairs performed as in FIG. 7, Section A. Each mixture contains between four and twelve molecules with shared chemical features, delivered at a final concentration of 10 μM per molecule. The color of each square (n=3, total cells in experiment >50,000) indicates the percentage of cells that responded, with only statistically significant response proportions colored. Mixture-MS4A pairs selected for deconvolution are marked with red circles. Deconvolution of selected odorant mixtures identifies monomolecular compounds that specifically activate each MS4A receptor. Individual odors delivered at 50 μM in liquid phase (n=3, total cells in experiment >68,000) to cells co-expressing GCaMP6s and the indicated MS4A receptor or GCaMP6s alone (FIG. 5, Section A-C). The aggregate percent of cells that responded to each chemical across three independent experiments is color-mapped as in FIG. 5, Section B. FIG. 6, Section A shows representative confocal images of HEK293 cells transfected with plasmids encoding GCaMP6S (green) and N-terminal mCherry-fusion proteins of the indicated MS4A protein (red), revealing the presence of mCherry-MS4A fusions at the plasma membrane. HEK293 cells transfected with GCaMP6s (green) and either mCherry alone or mCherry-MS4A6C (red) were immunostained under non-permeablizing conditions with an extracellularly-directed anti-MS4A6C antibody, revealing specific labeling of MS4A6C proteins (blue) indicating that MS4A6C is efficiently trafficked to the plasma membrane and adopts the predicted topology (FIG. 6, Section B). Deconvolution of selected odorant mixtures reveals monomolecular compounds that specifically activate each MS4A receptor. Individual odors were delivered at 50 □M in liquid phase to cells co-expressing GCaMP6s and the indicated MS4A receptor or mOR or GCaMP6s alone (FIG. 6, Section C). The aggregate percent of cells that responded to each chemical across three independent experiments is color-mapped as indicated, where only statistically significant responses were plotted. Traces of GCaMP6s fluorescence versus time averaged across all cells that responded to the best monomolecular odorant for each MS4A/mixture pair (FIG. 6, Section D).

### Example 4: Multiple Ms4a Genes are Expressed in Each Necklace Sensory Neuron

Representative images from single molecule fluorescent in situ hybridization (via RNAScope) of dissociated olfactory epithelial cells is shown. Puncta from probes against Ms4a family member are in red. Necklace cells were identified via co-labeling with an antibody against Car2 (blue) and GFP from the Gucy2d-IRES-TauGFP allele (green) or an RNAscope probe against a necklace marker gene (green, all panels except the top left). Necklace cells are not marked by a probe against the conventional OR gene Olfr151 (FIG. 7, Section A). Proportion of necklace OSNs (identified as Car2+) with two or more fluorescent puncta for each Ms4a (blue bars) and Olfr probe (red bars, including Ms4a puncta in OR174-9-IRES-GFP expressing cells; n=3 experiments, between 150-750 cells/probe, error bars are standard error of the proportion). Dashed red line represents the average value of negative controls. All Ms4a probes (other than negative controls Ms4a1, Ms4a2, Ms4a5) give a significantly higher proportion of positive cells than negative controls (p<0.01, one-tailed Z test on population proportions) (FIG. 7, Section B). Representative images of Car2+(blue) cells labeled with probes against the two indicated Ms4a family members are shown in FIG. 7, Section C. The proportion of cells with multiple puncta for neither, one, or both colors was quantified. The total number of cells in each category is shown in parentheses next to the proportion. Each pair shows significantly more double-positive cells (yellow) than expected if the two probes are independent (p<0.05, Fisher's Exact Test on 2×2 table).

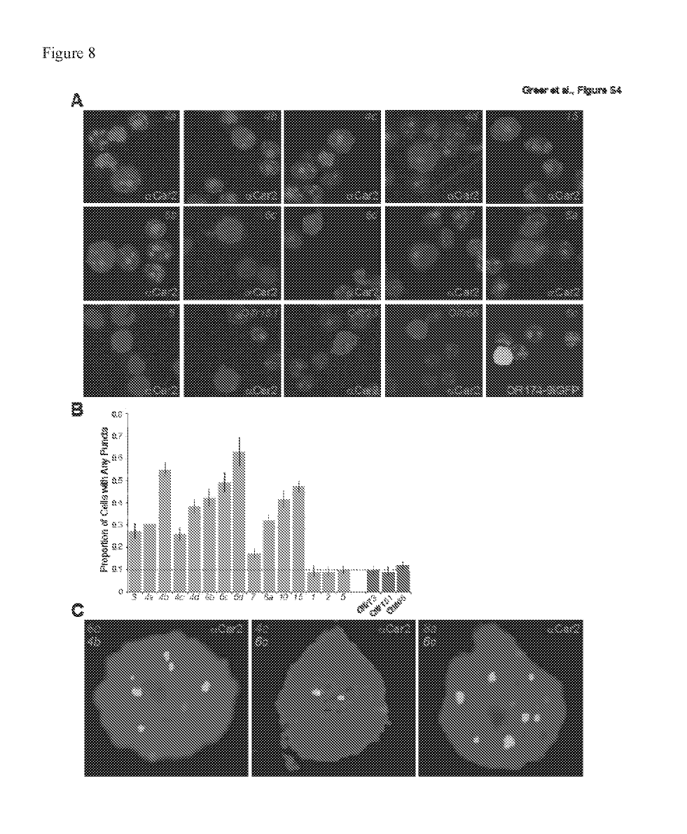

FIG. 8, Section A shows RNAscope assays of dissociated olfactory epithelial cells. Necklace cells were identified with an antibody against Car2 (blue), and puncta from probes against an Ms4a or Olfr family member are in red. Ms4a6c puncta are not found in GFP+ cells from dissociated OR174-IRES-GFP epithelia (bottom right). A proportion of necklace OSNs (identified as Car2+) with one or more fluorescent puncta for each Ms4a and Olfr probe (n=3 experiments, between 150-750 cells/probe, error bars are standard error of the proportion) are shown in FIG. 8, Section B. Dashed red line represents the average value of negative controls (Ms4a1, Ms4a2, Ms4a5, and the Olfr genes). Representative images of Car2+(blue) cells co-labeled with additional Ms4a probe pairs are shown in FIG. 8, Section C.

### Example 5: Multiple MS4A Proteins are Expressed within Necklace Sensory Endings and Glomeruli

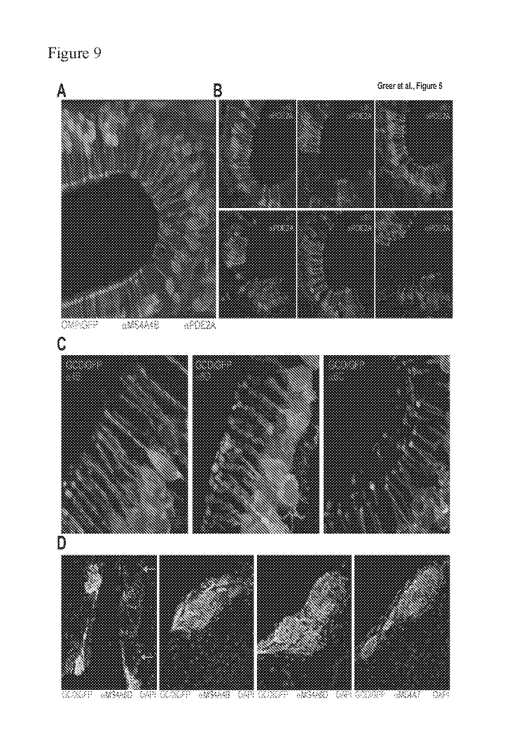

Anti-MS4A4B antibody stains every anti-PDE2A+ cell but does not stain OMP-IRES-GFP+ cells in sections of the olfactory epithelium (FIG. 9, Section A). FIG. 9, Section B shows immunostaining with antibodies against five different MS4A family members, each of which stains >95% of anti-PDE2A+ necklace cells in epithelial cul-de-sacs. In contrast, an antibody against MS4A5, which is not detected at the mRNA level in GC-D cells, does not label necklace cells. This antibody labels cells heterologously expressing mouse MS4A5 (data not shown). High resolution imaging of GCD-IRES-TauGFP+ necklace sensory neurons demonstrates anti-MS4A antibody labeling of dendritic knobs (FIG. 9, Section C). Anti-MS4A6D staining overlaps with all GCD-IRES-TauGFP+ necklace glomeruli in sections of the olfactory bulb. Blue arrows mark non-necklace glomeruli, which are not stained by anti-MS4A6D antibody. Similarly, anti-MS4A4B and anti-MS4A7 antibodies label each necklace glomerulus (FIG. 9, Section D).

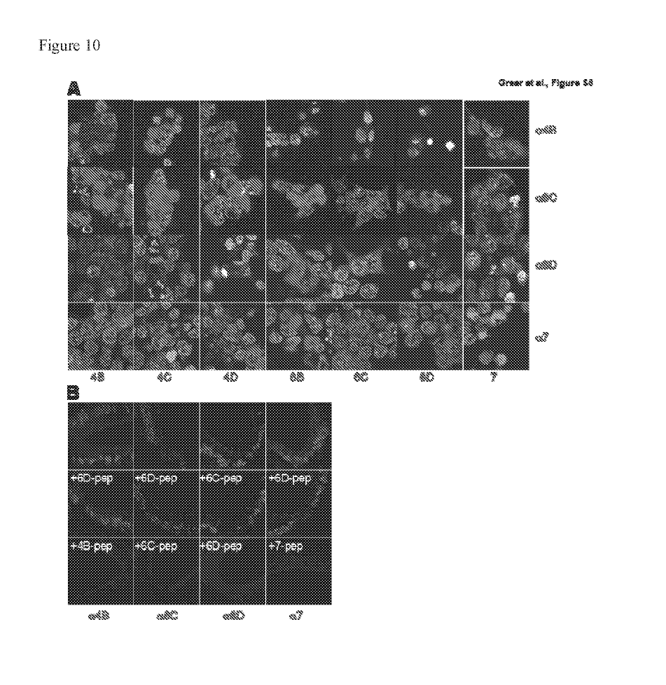

HEK293T cells transfected with a plasmid encoding a single mCherry-MS4A fusion protein and stained with the indicated anti-MS4A antibody, antibodies are specific, although under conditions of overexpression anti-MS4A4B cross-reacts modestly with the closely related MS4A4C, as does anti-MS4A6C with MS4A6B (FIG. 10, Section A). Representative images of cul-de-sac tissue sections immunostained in the presence of peptide competitor (1000-fold molar excess) are shown in FIG. 10, Section B. Only the antigenic peptide, and not a peptide from a different MS4A protein, blocks staining of necklace cells by a given antibody.

### Example 6: Multiple In Vitro MS4A Ligands Activate Single Necklace Cells In Vivo

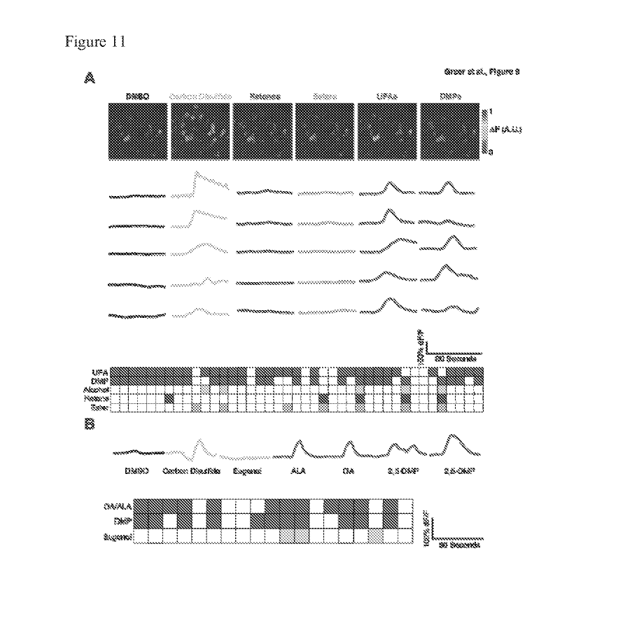

FIG. 11, Section A shows the cul-de-sac regions of intact olfactory epithelia from Emx1-cre;GCaMP3 mice were imaged and GCaMP fluorescence monitored as the epithelia were exposed to the indicated odorant mixtures in liquid phase. Representative heat-mapped fluorescent images (top row) and extracted fluorescent traces of CS2-responsive cells (middle rows) in response to the indicated odorants. Note that in this particular experiment responses to CS2 were larger than to DMPs and UFAs, but in general necklace cells responded with similar magnitude to these stimuli (see traces, which are from multiple, representative experiments). The responses of 41 cells (columns) to odor mixtures across five experiments were quantified (bottom row). All odorants were delivered at 100 μM each, (DMP: 2,3-DMP and 2,5-DMP, UFA: OA and ALA, ketones, esters, and alcohols as in Table 3 below).

TABLE 3
List of odorants used for functional imaging experiments

| | |
|---|---|
| Alcohols | 1-butanol, 2,5-dimethylphenol, eugenol, |
| | guaicol, 1-hexanol, isoeugenol, 1- |
| | nonanol, 1-octanol, 2-phenylethanol, |
| | thymol |
| Ketones | acetylanilone, acetophenone, 2-butanone, |
| | cyclohexanone, 3-decanone, |
| | dodecanolactone, 4-heptanone, 2- |
| | octanone, 2-pentanone, vanillin |
| Sulfurs | 2,4,5-trimethyl thiazole, TMT, thiophene, |
| | tetrahydrothiophene |
| Acids | formic acid, hexanoic acid, heptanoic |
| | acid, ocantoic acid, tiglic acid, valeric |
| | acid, isovaleric acid |
| Esters | allyl cinnamate, amyl acetate, benzyl |
| | acetate, cycohexyl aceatate, ethyl |
| | benzoate, ethyl propionate, ethyl valerate, |
| | ethyl tiglate, piperidine, propyl butyrate |
| Aldehydes | p-anise aldehyde, butyl formate, |
| | butyraldehyde, benzaldehyde, |
| | cinnamaldehyde, ethyl formate, heptanal, |
| | octanal, propionaldehyde, heptaldehdye |
| Nitrogenous | 2,5-dimethylpyrazine, 2,6- |
| | dimethylpyrazine, 2,3-dimethylpyrazine, |
| | indole, nicotine, pyrrolidine, pyridine, |
| | quinoline |
| Steroids | 4-Androsten-17alpha-ol-3-one sulphate, |
| | 5-Androsten-3Beta 17Beta-diol |
| | disulphate, 1,3,5(10)-Estratrien-3 17Beta- |
| | diol disulphate, 1,3,5(10)-Estratrien-3 |
| | 17alpha-diol 3-sulphate, 5alpha-pregnen- |
| | 3alpha-ol-20-one sulphate, 5beta- |
| | pregnen-3beta-ol-20-one sulphate, 4- |
| | pregnan-11beta 21-diol-3 20-dione 21- |
| | sulphate, 4-pregnen-21-ol-3 20-ione |
| | glucosiduronate, 1,3,5(10)-Estratrien-3 |
| | 17Beta-diol 3-sulphate, 4-pregnen-11beta |
| | 17,21-triol3 20-dione 21-sulphate |
| PUFAs | arachidonic acid, docosohexanoic acid, |
| | linoleic acid, linolenic acid, nervonic |
| | acid, oleic acid, petroselenic acid |
| Saturated fatty | decanoic acid, docosanoic acid, |
| acids | dodecanoic acid, eicosanoic acid, |
| | hexanoic acid, myristic acid, |
| | octadecanoic acid, octanoic acid, palmitic |
| | acid |
| Terpenes | R-carvone, 1,4-cineole, citral, |
| | cintronellal, R-fenchone, E-beta |
| | farnesene, geraniol, alpha-ionone, |
| | linalool, +-menthone, gamma-terpinene, |
| | 1,3-minus-verbenone |

DMPs and UFAs each activated significantly more cells than all negative controls (P<0.001. Fisher's Exact Test. corrected for multiple comparisons). Significantly more cells responded to both UFA and DMP than expected by chance (P<0.01, Fisher's Exact Test). FIG. 11, Section B shows fluorescent traces extracted from a necklace cell in response to the indicated monomolecular odorant (top row). The responses of 20 cells (columns) to at least one chemical within the indicated class across six experiments were quantified (bottom row). All odorants were delivered at 100 μM each. DMPs and UFAs each activated significantly more cells than all negative controls (P<0.01, Fisher's Exact Test, corrected for multiple comparisons). Significantly more cells responded to both UFA and DMP than expected by chance (P<0.01, Fisher's exact test). Scale bar indicates time on the X-axis and relative fluorescence on the Y-axis.

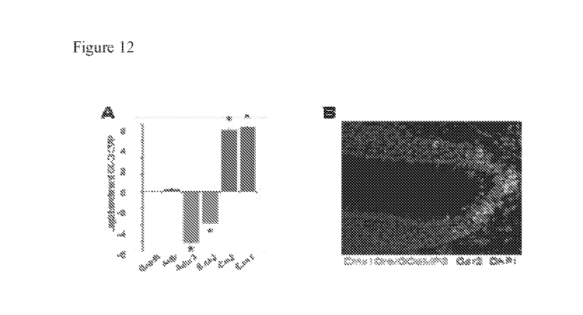

FIG. 12, Section A shows the quantification of mRNA expression in GC-D cells relative to OMP cells using the single-molecule detection method Nanostring. 10,000 GFP positive cells from Gucy2dIRFSGFP or OmpGFP mice were sorted into Trizol and the RNA was isolated as described above. Three biological replicate RNA samples were hybridized to Nanostring probes using nCounter Elements reagents according to the manufacturer's specifications. The protocol was modified to perform the hybridization step at 67° C. for 48 hours to maximize the detection of low abundance transcripts. RNA molecules that hybridized to probe were captured and quantified using an automated Nanostring prep station following the manufacturer's instructions. The resultant data were analyzed using nSolver software. Briefly, the average number of detected molecules for six internal negative control probes (whose complementary sequences are not present in the mouse genome) was used to calculate a rate of non-specific hybridization. After subtracting the amount of binding resulting from non-specific interactions, the number of molecules of each RNA transcript found in GC-D samples and OMP samples was compared using Student's t-test. See table 2 for probe sequences used in these experiments. A list of probes sequences used in the Nanostring experiments can be found in Table 2 below.

TABLE 2
List of probe sequences used in
Nanostring experiments

| | |
|---|---|
| Ms4a1 | GCAACCTGCTCCAAAAGTGAACCTCAAAAGGACATCTTCACT |
| | GGTGGGCCCCACACAAAGCTTCTTCATGAGGGAATCAAAGGC |
| | TTTGGGGGCTGTCCAA |
| Ms4a2 | ACAGAAAATAGGAGCAGAGCAGATCTTGCTCTCCCAAATCCA |
| | CAAGAATCCTCCAGTGCACCTGACATTGAACTCTTGGAAGCA |
| | TCTCCTGCCAAAGCAG |
| Ms4a3 | CCAGGCTTTCAAGGGTTGCCAATCTTCACCGTCACCTGATGT |
| | CTGCATTTCCCTGGGTTCCTCATCAGATGGCCTGGTGTCTTT |
| | AATGCTGATTCTCACC |
| Ms4a4a | AACCCAAAATCCTTGGGATTGTGCAGATTGTAATCGCCATCA |
| | TGAACCTCAGCATAGGAATTATGATGATAATTGCCACTGTGT |
| | CGACCGGTGAAATACC |
| Ms4a4b | CCTAGGATATTAACACTTCATTGCACTGGCTTTTGAGGTGAA |
| | TATTAGATTTACTGTAAGTATGTAAGTCAAGCACTTATTAGG |
| | TCAACAACACTTCAAC |
| Ms4a4c | TGGCAAATCTATCTTCTGAACCACTCATTTCTGTGGTCTTAA |
| | TGGCTCCAATTTGGGGACCAATAATGTTCATTGTCTCAGGAT |
| | CCCTGTCAATTGCAGC |
| Ms4a4d | ACAACTGGCACTACCATCGTGGTGAAAACCCAGCTCAAGCAT |
| | ACCCACAAATAGAGTCCCACATCGAAACTCCACCACATTACT |
| | CAAGGATACTGTTTCT |
| Ms4a5 | TGAATTTACTTAGTGCTCTGGGAGCAGCAGCTGGAATCATTC |
| | TCCTCATATTTGGCTTCCTTCTAGATGGGGAATTCATCTGTG |
| | GCTATTCTCCAGATGG |
| Ms4a6b | AAACAAAACTAAATACCACAAAAACAAATGGAACTATACCGC |
| | AGAAGATATGTCTTCATGATAATGCAGAAATTCCAACCATCA |
| | CAGGGTAGCAATGCTT |
| Ms4a6c | CATGATTCCACAGGTAGTGACCAATGAGACCATCACAACGAT |
| | TTCACCAAATGGAATCAACTTTCCCCAAAAAGACGAGTCCCA |
| | GCCTACCCAACAGAGG |
| Ms4a6d | AGTTTGGCTGCTTTAGAGCCTGCCTTGCAGCAATGTAAGCTG |
| | GCTTTCACACAACTAGACACAACCCAAGATGCTTATCATTTC |
| | TTTAGCCCTGAGCCAT |
| Ms4a7 | GCCTCCAATGTAGCAAGCTCTGTTGTTGCCGTCATTGGCCTC |
| | TTCCTCTTCACCTATTGTCTGATAGCCCTGGGGAGTGCTTTC |
| | CCACACTGTAACTCAG |
| Ms4a8a | TGTCACTACAACAATCCAGGTGTGGTCATTCCAAATGTCTAT |
| | GCAGCAAACCCAGTGGTCATCCCAGAACCACCAAACCCAATA |
| | CCAAGTTATTCCGAAG |
| Ms4a10 | CCTAAGACCTCTCTGAAGGTTCTCTGTGTGATAGCCAACGTT |
| | ATCAGCTTGTTCTGCGCACTGGCCGGCTTCTTTGTCATTGCC |
| | AAGGACCTCTTCCTGG |
| Ms4a13 | TTTCATGGCTGCTAACACCTGATGTAGGTGCCCATGAGATTC |
| | CCATATAACAAGGCACACCTCATGCATTTTGTGCAAAAGGAA |
| | ATTCACAACAAGGTGA |
| Ms4a15 | GTGGGAAATCTTGGCTTCGCAGAGGTTTCGGAGGTTTGTCTT |
| | CAAGATCATTAAGCACGGAGAACTCAGAATGTTCCAGAATAG |
| | ACTGGCATTTCAGAGG |
| Ms4a18 | GAATTCATCCTCACCTGCATAGCCTCACATTTTGGATGCCAG |
| | GCTGTCTGCTGCGCCCATTTTCAGAACATGACAATGTTCCCA |
| | ACCATATTTGGTGGCA |
| Pde1c | GCTGTAATCGATGCATTGAAGGATGTGGATACGTGGTCCTTC |
| | GATGTCTTTTCCCTCAATGAGGCCAGTGGAGATCATGCACTG |
| | AAGTTCATTTTCTATG |
| Adcy3 | CAACAACGGCGGCATCGAGTGTCTACGCTTCCTCAATGAGAT |
| | CATCTCTGATTTTGACTCTCTCCTGGACAATCCCAAATTCCG |
| | GGTCATCACCAAGATC |
| Cnga2 | GCTTGTGGATAATGGAGATCATGTGGGTTGAATTTCTAAGAG |
| | CGTGACCTCCTAAGTCTCACAAGGAATCAGAGAATAGCTAAA |
| | TTGTCCTTCCTGAGGC |
| Actb | CAGGTCATCACTATTGGCAACGAGCGGTTCCGATGCCCTGAG |
| | GCTCTTTTCCAGCCTTCCTTCTTGGGTATGGAATCCTGTGGC |
| | ATCCATGAAACTACAT |
| Gapdh | AGGTTGTCTCCTGCGACTTCAACAGCAACTCCCACTCTTCCA |
| | CCTTCGATGCCGGGGCTGGCATTGCTCTCAATGACAACTTTG |
| | TCAAGCTCATTTCCTG |
| Pde2a | CCACTAGCTTCTCTTCTGTTTTGTTCCCTATGTGTCGTGGGT |
| | GGGGGAGGGGGCCACCTGCCTTACCTACTCTGAGTTGCCTTT |
| | AGAGAGATGCATTTTT |
| Car2 | TGCCCAGCATGACCCTGCCCTACAGCCTCTGCTCATATCTTA |
| | TGATAAAGCTGCGTCCAAGAGCATTGTCAACAACGGCCACTC |
| | CTTTAACGTTGAGTTT |
| Golf | ATCGAAGACTATTTCCCGGAGTATGCCAATTATACTGTCCCT |
| | GAAGATGCAACACCAGATGCGGGAGAAGATCCCAAAGTTACA |
| | AGAGCAAAGTTCTTTA |
| Emx1 | CAGGCAAGCGACGTTCCCCAGGACGGGCTGCTTTTGCACGGG |
| | CCCTTCGCACGCAAGCCCAAGCGGATTCGCACAGCCTTCTCG |
| | CCCTCGCAGCTGCTGC |

Marker genes for OMP cells such as Adcy3 (green bars) and GC-D cells like Car2 (red) are enriched in the appropriate populations. This analysis revealed that whereas OMP cells express the transcription factor Emx2, GC-D sensory cells exclusively express Emx1* p<0.05, paired t-test. FIG. 12, Section B shows immunohistochemical analysis of sections prepared from the nasal epithelium of mice co-expressing an Emx1-cre allele and a Cre-dependent GCaMP3 reporter using antibodies against GCaMP (green) and the necklace marker CAR2 (red) reveals that a large fraction of GCaMP-expressing cells are necklace cells, note that CAR2 staining tends to be enriched in nuclei whereas GCaMP is enriched.

### Example 7: MS4A Ligands Activate Necklace Sensory Neurons, and MS4A Proteins Confer Responses to Conventional Olfactory Sensory Neurons in Awake, Behaving Mice

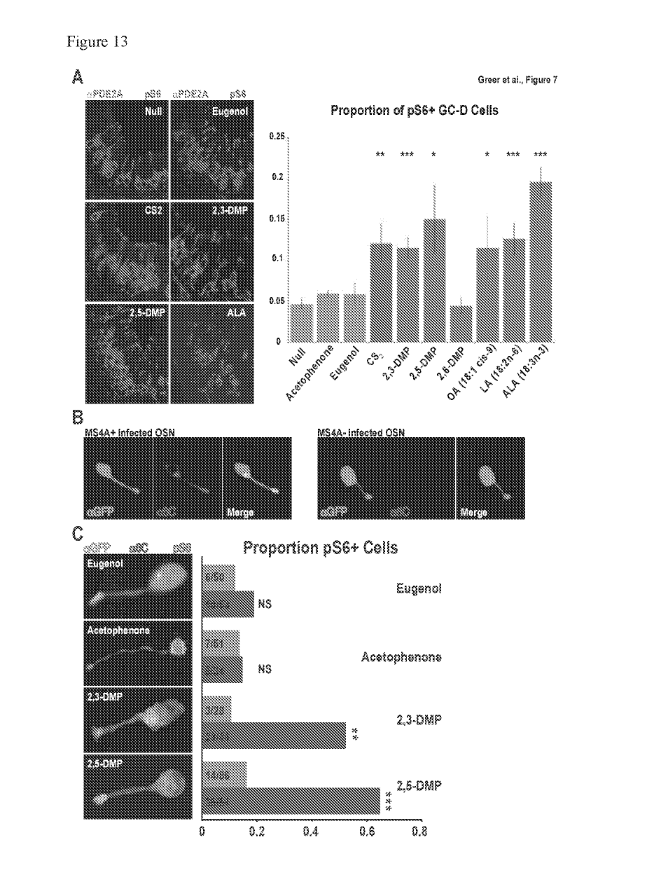

FIG. 13, Section A shows example images of cul-de-sacs from mice exposed to the indicated odorant, immunostained for the necklace cell marker PDE2A (blue) and the neuronal activity marker phospho-S6 (pSerine240/244) (red) (left panels). Quantification of the proportion of pS6+ necklace cells in mice exposed to each odorant (right panel, mean+/−SEM, n>=3 independent experiments, * indicates p<0.05, ** indicates p<0.01, and *** indicates p<0.0001, unpaired t-test compared to null exposure). FIG. 13, Section A shows olfactory epithelial sections of mice infected with adenovirus carrying an Ms4a6c-IRES-GFP expression cassette reveal that a subset of virally infected cells (green) also express MS4A6C protein (red). FIG. 13, Section C shows representative images (left panels) and quantification (right panel) of phospho-S6 positive, virally infected OSNs exposed to the indicated odorant. Gray bars: GFP-positive/MS4A6C-negative, red bars: GFP-positive/MS4A6C-positive cells (n>=3 animals per odor, ** indicates p<0.001, ***p<0.0001, Fisher's Exact Test comparing MS4A6C-positive to MS4A6C-negative cells for each odorant).

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments are described herein. Such equivalents are intended to be encompassed by the following claims.

1. A method of treating or preventing Alzheimer's disease in a subject comprising administering to the subject an agent that modulates the activity of an MS4A receptor.

2. The method of claim 1, wherein the MS4A receptor is an MS4A4 or a homolog thereof, an MS4A6 or a homolog thereof, or an MS4A7 or a homolog thereof.

3-4. (canceled)

5. The method of claim 1, wherein the agent activates the MS4A receptor.

6. The method of claim 1, wherein the agent inhibits activity of the MS4A receptor.

7. The method of claim 1, wherein the agent is a small molecule, a polypeptide, or a polynucleotide, an inhibitory polynucleotide, or an antibody.

8-9. (canceled)

10. The method of claim 7, wherein the agent is an small molecule and the small molecule is selected from 2,5-dimethylpyrazine, 3-aminopyrazine (3-AP), and tetramethylpyrazine.

11-12. (canceled)

13. The method of claim 7, wherein the agent is a polypeptide and the polypeptide is an MS4A protein or a fragment thereof or an MS4A receptor ligand or fragment thereof.

14-16. (canceled)

17. The method of claim 1, wherein the agent is an inhibitory polynucleotide specific for an MS4A receptor, and the inhibitory polynucleotide is selected from the group consisting of siRNA, shRNA, and an antisense RNA molecule, or a polynucleotide that encodes a molecule selected from the group consisting of siRNA, shRNA, and/or an antisense RNA molecule.

18-55. (canceled)

56. A method of treating or preventing an allergy and/or atopy, in a subject comprising administering to the subject an agent that modulates the activity of an MS4A receptor.

57. The method of claim 56, wherein the MS4A receptor is MS4A2 or a homolog thereof.

59-61. (canceled)

62. The method of claim 56, wherein the agent is a small molecule, a polypeptide, a polypeptide, or a polynucleotide, an inhibitory polynucleotide, or an antibody.

63-70. (canceled)

71. The method of claim 56, wherein the agent is an inhibitory polynucleotide specific for an MS4A receptor, and the inhibitory polynucleotide is selected from the group consisting of siRNA, shRNA, and an antisense RNA molecule, or a polynucleotide that encodes a molecule selected from the group consisting of siRNA, shRNA, and/or an antisense RNA molecule.

72-73. (canceled)

74. A method of treating or preventing asthma in a subject comprising administering to the subject an agent that modulates the activity of an MS4A receptor.

75. The method of claim 74, wherein the MS4A receptor is MS4A2 or a homolog thereof.

76. The method of claim 74, wherein the agent activates the MS4A receptor.

77. The method of claim 74, wherein the agent inhibits activity of the MS4A receptor.

78. The method of claim 74, wherein the agent is a small molecule, a polypeptide, a polypeptide, or a polynucleotide, an inhibitory polynucleotide, or an antibody.

79-83. (canceled)

84. The method of claim 83, wherein the polypeptide is an MS4A protein or a fragment thereof or an MS4A receptor ligand or fragment thereof.

85-87. (canceled)

88. The method of claim 74, wherein the agent is an inhibitory polynucleotide specific for an MS4A receptor and the inhibitory polynucleotide is selected from the group consisting of siRNA, shRNA, and an antisense RNA molecule, or a polynucleotide that encodes a molecule selected from the group consisting of siRNA, shRNA, and/or an antisense RNA molecule.

89-216. (canceled)
